(19) United States
(12) Patent Application Publication
(10) Pub. No.: US 2005/0158724 A1
(43) Pub. Date: Jul. 21, 2005

(54) Methods, of identifying compounds that modulate a dna repair pathway and/or retroviral infectivity, the compounds, and uses thereof

(76) Inventors: Richard A. Fishel, Penn Valley, PA (US); Kristine E. Yoder, Penn Valley, PA (US)

Correspondence Address: Suzanne E Miller;Woodcock Washburn, 46th Floor, One Liberty Place, Philadelphia, PA, 19103, US

(21) Appl. No.: 10/510,363

(22) PCT Filed: Apr. 4, 2003
(86) PCT No.: PCT/US03/10302

Related U.S. Application Data
(60) Provisional application No. 60/370,376, filed on Apr. 5, 2002.

(51) Int. Cl. C12Q 1/68; C12N 15/867

(52) U.S. Cl. 435/6; 435/456

(57) ABSTRACT

Retroviruses are RNA viruses that must insert a DNA copy (cDNA) of their genome into the host genome in order to carryout a productive infection. One host cellular pathway that defends against retroviral cDNA integration involves highly conserved proteins of a host DNA repair pathway. These proteins represent novel targets for anti-retroviral drugs. The invention presented herein provides, inter alia, methods of identifying compounds that induce a DNA repair pathway and/or inhibit retroviral cDNA integration into a host genome, compounds thus identified, uses of such compounds, and kits for identifying and testing of the efficacy of compounds in inducing a DNA repair pathway, inhibiting retroviral cDNA integration, and inhibiting retroviral infection.

### CROSS REFERENCE TO RELATED APPLICATIONS

This application claims the benefit of U.S. Provisional Application No. 60/370,376, filed Apr. 5, 2002, which is hereby incorporated by reference in its entirety.

### STATEMENT REGARDING FEDERALLY SPONSORED RESEARCH

This work was supported in part by a research grant from the National Institutes of Health, grant number GM62556. The United States Government may have certain rights in this invention.

### FIELD OF THE INVENTION

The present invention is directed, in part, to methods for inducing a DNA repair pathway, methods for identifying compounds that induce a DNA repair pathway and/or inhibit retroviral infectivity, methods of treating a condition caused by a retroviral infection with compounds that induce a DNA repair pathway and/or inhibit retroviral cDNA integration into the host cell genome, methods for inhibiting a DNA repair pathway and/or increasing retroviral cDNA integration, methods for identifying compounds that inhibit a DNA repair pathway and/or increase retroviral infectivity, and methods of treating a condition by improving gene delivery with compounds that inhibit a DNA repair pathway and/or increase retroviral cDNA integration into the host cell genome.

### BACKGROUND OF THE INVENTION

Retroviruses are RNA viruses that must insert a DNA copy (retroviral cDNA) of their genome into the host chromosome in order to carry out a productive infection. Retroviral integration can result in mutagenic inactivation of genes at the sites of cDNA insertion or in aberrant expression of adjacent host genes, both of which can have deleterious consequences for the host organism. Furthermore, retroviruses present considerable risk to human and animal health, as evidenced by the fact that retroviruses cause diseases such as, but not limited to, acquired immune deficiency syndrome (AIDS, caused by human immunodeficiency virus, HIV-1), various animal cancers, feline immunodeficiency virus (FIV), and human adult T-cell leukemia/lymphoma. Retroviruses also have been associated with other common disorders, including, but not limited to, Type I diabetes and multiple sclerosis.

Recent efforts to combat such retroviral-borne diseases have focused on the identification of inhibitors of retroviral proteins involved in infection. Two mechanisms characterize the mode of infection of retroviruses: reverse transcription and integration (Coffin, J. M., S. H. Hughes, and H. E. Varmus. RETROVIRUSES. Cold Spring Harbor, N.Y.: Cold Spring Harbor Laboratory Press, 1997). Both processes are essential for retroviruses to productively infect a cell (Tisdale, M., T. Schulze, B. A. Larder, and K. Moelling. Mutations within the RNase H domain of human immunodeficiency virus type 1 reverse transcriptase abolish virus infectivity. Journal of General Virology, 72: 59-66, 1991; LaFemina, R. L., C. L. Schneider, H. L. Robbins, P. O. Callahan, K. LeGrow, E. Roth, W. A. Schleif, and E. A. E. Emini. Requirement of active human immunodeficiency virus type 1 integrase enzyme for productive infection of human T-lymphoid cells. Journal of Virology, 66: 7414-7419, 1992; Sakai, H., M. Kawamura, J. Sakuragi, S. Sakurgai, R. Shibata, A. Ishimoto, N. Ono, S. Ueda, and A. Adachi. Integration is essential for efficient gene expression of human immunodeficiency virus type 1. Journal of Virology, 67: 1169-1174, 1993; Englund, G., T. S. Theodore, E. O. Freed, A. Engleman, and M. A. Martin. Integration is required for productive infection of monocyte-derived macrophages by human immunodeficiency virus type 1. Journal of Virology, 69: 3216-3219, 1995). To date, most drug development programs have focused on inhibition of virally encoded products, including retroviral reverse transcriptases and proteases. However, given the short life cycle of retroviruses and their inherently high rates of genetic change or mutation, such strategies result in the development of drug resistant virus derivatives through alterations of the virally encoded target molecules. Thus, most anti-retroviral drugs that interfere with virally encoded proteins are effective, if at all, for only limited periods of time. Another limitation of drugs that target retrovirus proteins is that many do not have broad applicability and are highly specific to a particular virus or even a certain strain of a particular virus.

As an example of the limitations of present retroviral therapies that target retroviral proteins, a current treatment for AIDS, caused by the HIV retrovirus, consists of a cocktail of three or four anti-retroviral drugs termed HAART (highly active anti-retroviral therapy) (Autran, B., G. Carcelain, T. S. Li, C. Blanc, D. Mathez, R. Tubiana, C. Katlama, P. Debre, and J. Leibowitch. Positive effects of combined antiretroviral therapy on CD4+ T cell homeostasis and function in advanced HIV disease. Science, 277: 112-116, 1997). The retroviral reverse transcriptase is inhibited by two families of HAART drug components, nucleotide analogs and non-nucleotide inhibitors. The remaining drugs used in HAART are retroviral protease inhibitors, which target another HIV enzyme. However, 78% of new HIV infections are resistant to at least one HAART drug component, and an effective HIV vaccine has not been developed (Richman, D. In: INTERSCIENCE CONFERENCE ON ANTIMICROBIAL AGENTS AND CHEMOTHERAPY, Chicago, Ill., 2001; Cohen, J. Debate begins over new vaccine trials. Science, 293: 1973, 2001). Furthermore, most of the identified drugs that inhibit the retroviral integrase enzyme of HIV have been unsuccessful in human trials due to lack of specificity or poor bioavailability (Craigie, R. HIV integrase, a brief overview from chemistry to therapeutics. Journal of Biological Chemistry, 276: 23213-23216, 2001; Hazuda, D. J., P. Felock, M. Witmer, A. Wolfe, K. Stillmock, J. A. Grobler, A. Espeseth, L. Gabryelski, W. Schleif, C. Blau, and M. D. Miller. Inhibitors of strand transfer that prevent integration and inhibit HIV-1 replication in cells. Science, 287: 646-650, 2000). Thus, the development of novel HIV infection and AIDS therapeutics is critical. Also of great importance is the development of an effective HIV vaccine.

Retroviruses also are used for gene delivery and are likely to play increasingly important roles in gene therapy. Accordingly, methods and compounds that increase retroviral cDNA integration into the host genome, and hence increase gene delivery, are of great importance.

Thus, an understanding of how retroviruses function and how they can be controlled is of great commercial and medical importance. Such an understanding would allow the development of novel strategies for treating retroviral infection and for improving gene delivery in gene therapy methodologies.

The present invention elucidates a pathway of DNA repair and its components involved in retrovirus infection and by providing, inter alia, methods and assay systems for identifying compounds that inhibit retroviral cDNA integration and/or induce a DNA repair pathway, methods for inducing a DNA repair pathway and/or inhibiting retroviral cDNA integration, methods of treating a retroviral infection with compounds that induce a DNA repair pathway and/or inhibit retroviral cDNA integration into the host cell genome, methods for inhibiting a DNA repair pathway and/or increasing retroviral cDNA integration, methods for identifying compounds that inhibit a DNA repair pathway and/or increase retroviral infectivity, and methods of treating a condition by improving gene delivery with compounds that inhibit a DNA repair pathway and/or increase retroviral cDNA integration into the host cell genome.

The stimulation of an intrinsic host defense mechanism as presented herein is a valuable addition to the treatment of HIV, or any other retrovirus, infection. First, it is very difficult or impossible for the retrovirus to mutate in such a way that it evades drug action. Host cell factors are not subject to the highly mutagenic viral replication process, the foundation for development of retroviral drug resistance. Second, since integration is a prerequisite for all retroviruses to be infective, drugs that induce the formation of 1-LTR or 2-LTR circles are effective against a wide spectrum of retrovirus types. Furthermore, little toxicity is associated with this form of treatment since it is an endogenous system (i.e., host cell factors) that is stimulated. The treatment for retroviral infections presented herein is anticipated to be used in combination with other currently available antiviral drugs, for example, as part of HAART.

### SUMMARY OF THE INVENTION

In one embodiment of the invention, methods for identifying compounds that inhibit retroviral cDNA integration by contacting a cell or cell extract with a non-circularized retroviral cDNA in the presence of a test compound; contacting a cell or cell extract of the same type with a non-circularized retroviral cDNA in the absence of a test compound; and determining whether the amount of retroviral cDNA circularization is increased in the presence of the test compound relative to the level of retroviral cDNA circularization that occurs in the absence of the test compound are provided.

In another embodiment of the invention, methods for identifying compounds that inhibit retroviral cDNA integration by contacting a cell or cell extract with a non-circularized retroviral cDNA in the presence of a test compound and determining the amount of retroviral cDNA circularization are provided.

One aspect of the present invention provides methods for identifying compounds that induce a DNA repair pathway in a cell by contacting at least one component of a DNA repair pathway with a non-circularized retroviral cDNA in the presence of a test compound; contacting the component of the DNA repair pathway with a non-circularized retroviral cDNA in the absence of the test compound; and determining whether the amount of retroviral cDNA circularization is increased in the presence of the test compound relative to the amount of retroviral cDNA circularization that occurs in the absence of the test compound. The methods of the invention may be performed in a cell or in cell extract. Cells that may be employed by the methods of the invention, or from which cell extract may be derived, include, for example, mammalian, including for example human and chicken, yeast, and plant cells. The component of a DNA repair pathway that may be contacted or upregulated, either directly or indirectly, by the test compound includes, but is not limited to, at least one of nucleic acid molecules encoding XPA, XPB, XPC, XPD, XPE, XPF, XPG, RAD50, RAD52, RAD54, RAD57, RAD59, MSH2, CDC9, hRAD50, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer; polypeptides encoded thereby; and homologs thereof.

In some embodiments of the invention, at least one component of a DNA repair pathway exhibits reduced biological activity in the absence of the test compound relative to wild-type biological activity of the component in the absence of the test compound. The component exhibiting reduced biological activity includes, but is not limited to, at least one of nucleic acid molecules encoding XPA, XPB, XPC, XPD, XPE, XPF, XPG, RAD50, RAD52, RAD54, RAD57, RAD59, MSH2, CDC9, hRAD50, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer; polypeptides encoded thereby; and homologs thereof.

In some aspects of the invention, the retroviral cDNA contains at least one marker gene and at least one promoter such that the marker gene is expressed from the promoter upon retroviral cDNA circularization. An increase in retroviral cDNA circularization in the methods of the invention may be detected by an increase in the level of expression of the marker gene or in the level of activity of the polypeptide encoded by the marker gene in the presence of the test compound relative to the level thereof in the absence of the test compound. Examples of marker genes that may be used in the methods of the invention include, but are not limited to, genes encoding green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT). Examples of promoters that may be used in the methods of the invention include, but are not limited to, promoters derived from adenovirus, SV40, parvoviruses, vaccinia virus, cytomegalovirus, or mammalian genomic DNA, an MSH2 promoter, constitutive promoters including 3-phosphoglycerate kinase and various other glycolytic enzyme gene promoters, or inducible promoters including the alcohol dehydrogenase-2 promoter or metallothionine promoter.

Also provided herein are retroviral vectors having a nucleic acid molecule including a promoter and a marker gene that is expressed upon circularization of the nucleic acid molecule. In some embodiments of the invention, the retroviral vector has a nucleic acid sequence of SEQ ID NO:5 or SEQ ID NO:6.

In some aspects of the invention, compounds that induce a DNA repair pathway and/or inhibit retroviral cDNA integration into the genome of a host cell are provided. In some embodiments of the invention, compounds that prevent retroviral infection of the host cell are provided. In other aspects of the invention, compounds that inhibit a DNA repair pathway and/or increase retroviral cDNA integration are provided.

Some aspects of the invention are directed to pharmaceutical compositions of the compounds of the invention. Pharmaceutical compositions of the invention, for example for the treatment of a retroviral infection, contain a therapeutically effective amount of at least one compound identified according to the methods of the invention, or a pharmaceutically acceptable salt thereof, and a pharmaceutically acceptable excipient.

Additional embodiments of the invention are directed to methods of inducing a DNA repair pathway of a cell by administering at least one compound identified by the methods of the invention to the cell. In some aspects of the invention, the compound inhibits retroviral cDNA integration into the genome of the cell.

Some embodiments of the invention provide methods of treating a retroviral infection of a patient by administering at least one compound identified by the methods of the invention, or a pharmaceutical composition thereof, to the patient. The patient may be a plant or a mammal, including, but not limited to, avians, felines, canines, bovines, ovines, porcines, equines, rodents, simians, and humans. Examples of retroviral infections that may be treated according to the methods of the invention include, but are not limited to, retroviral infections associated with at least one condition of acquired immune deficiency syndrome (AIDS), human immunodeficiency virus (HIV-1) infection, cancer, human adult T-cell leukemia/lymphoma, FIV, Type I diabetes, and multiple sclerosis.

One aspect of the present invention provides methods for identifying compounds that inhibit a DNA repair pathway and/or increase retroviral cDNA integration in a cell by contacting at least one component of a DNA repair pathway with a non-circularized retroviral cDNA in the presence of a test compound; contacting the component of the DNA repair pathway with a non-circularized retroviral cDNA in the absence of the test compound; and determining whether the amount of retroviral cDNA circularization is increased in the presence of the test compound relative to the amount of retroviral cDNA circularization that occurs in the absence of the test compound. The methods of the invention may be performed in a cell or in cell extract. Cells that may be employed by the methods of the invention, or from which cell extract may be derived, include, for example, mammalian, including but not limited to human and chicken, yeast, and plant-cells. The component of a DNA repair pathway that may be contacted or upregulated, either directly or indirectly, by the test compound includes, but is not limited to, at least one of nucleic acid molecules encoding XPA, XPB, XPC, XPD, XPE, XPF, XPG, RAD50, RAD52, RAD54, RAD57, RAD59, MSH2, CDC9, hRAD50, hRAD51, HRAD51B, hRAD51C, hRAD51D, hXRCC2, hXRCC3, XRCC4, ligase IV, hMRE11, XRS2 (NBS1), DNA-PK, and Ku70/80 heterodimer; polypeptides encoded thereby; and homologs thereof.

In some aspects of the invention, the retroviral cDNA contains at least one marker gene and at least one promoter such that the marker gene is expressed from the promoter upon retroviral cDNA circularization. A decrease in retroviral cDNA circularization in the methods of the invention may be detected by a decrease in the level of expression of the marker gene or in the level of activity of the polypeptide encoded by the marker gene in the presence of the test compound relative to the level thereof in the absence of the test compound. Examples of marker genes that may be used in the methods of the invention include, but are not limited to, genes encoding green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT). Examples of promoters that may be used in the methods of the invention include, but are not limited to, promoters derived from adenovirus, SV40, parvoviruses, vaccinia virus, cytomegalovirus, or mammalian genomic DNA, an MSH2 promoter, constitutive promoters including 3-phosphoglycerate kinase and various other glycolytic enzyme gene promoters, or inducible promoters including the alcohol dehydrogenase-2 promoter or metallothionine promoter.

In some aspects of the invention, compounds that inhibit a DNA repair pathway and/or increase retroviral cDNA integration into the genome of a host cell are provided. In some embodiments of the invention, compounds identified according to the methods are provided.

Some aspects of the invention are directed to pharmaceutical compositions of the compounds of the invention. Pharmaceutical compositions of the invention, for example for improving the efficiency of gene delivery in a gene therapy, contain a therapeutically effective amount of at least one compound identified according to the methods of the invention, or a pharmaceutically acceptable salt thereof, and a pharmaceutically acceptable excipient.

Additional embodiments of the invention are directed to methods of inhibiting a DNA repair pathway and/or increasing retroviral cDNA integration of a cell by administering at least one compound identified by the methods of the invention to the cell.

Additional embodiments of the invention provide methods for increasing the efficiency of gene delivery in a gene therapy by administering a compound of the invention. The patient may be a plant or a mammal, including, but not limited to, avians, felines, canines, bovines, ovines, porcines, equines, rodents, simians, and humans.

Additional aspects of the invention provide assay systems for identifying compounds that induce a DNA repair pathway. In some aspects of the invention, a cell-free system for identifying a compound that induces a DNA repair pathway containing at least one component of a DNA repair pathway, noncircularized retroviral cDNA having a marker gene that is expressed upon retroviral cDNA circularization, and genomic DNA is provided. Also provided herein are cell-based systems for identifying a compound that induces a DNA repair pathway containing a retrovirus having a marker gene and a cell having at least one component of a DNA repair pathway. In some embodiments of the assay systems, the component of the DNA repair pathway exhibits reduced biological activity relative to wild-type biological activity of the component. In some embodiments of the invention are provided cell-based assay systems for identifying compounds that inhibit retroviral cDNA integration having a call and a retrovirus containing a circularization marker gene. Also encompassed within the scope of the invention are cell-free assay systems for identifying compounds that inhibit retroviral cDNA integration having host genomic DNA and noncircularized retroviral cDNA having a circularization marker gene.

Another aspect of the invention is kits containing a retrovirus or retroviral vector of the invention. Such kits may include conventional kit component(s) including but not limited to container(s), label(s), and instructions.

Other aspects of the invention include methods of screening for a compound which inhibits retroviral infectivity by exposing at least one component of a DNA repair pathway to a test compound; inducing DNA repair; measuring one of an amount of retroviral cDNA circularization wherein the circularization juxtaposes a promoter to a marker gene, and the physical recombination of retroviral cDNA; quantifying expression of the marker gene; inhibiting integration of the retroviral cDNA into a host cell genome; and identifying the compound. Also provided are methods of screening for a compound which inhibits retroviral infectivity by exposing a component of a DNA repair pathway to a test compound; inducing DNA repair; measuring one of an amount of retroviral cDNA circularization wherein the circularization juxtaposes a promoter to a marker gene, and the physical recombination of retroviral cDNA; measuring an amount of expression of the marker gene which is indicative of an increase in circularization; inhibiting integration of the retroviral cDNA into a host cell genome; and identifying the compound. The component of the DNA repair pathway may be at least one of XPB or XPD but is not limited to the XPB or XPD members of the DNA repair pathway. The component of the DNA repair pathway may be a gene in the DNA repair pathway and the compound which induces DNA repair may upregulate the gene so that DNA repair is induced and retroviral integration is inhibited. The component of the DNA repair pathway also may be a protein in the DNA repair pathway and the compound which induces DNA repair induces an activity or function of the protein so that DNA repair is induced and retroviral integration is inhibited. Additional embodiments of the invention include methods of inhibiting retroviral infectivity in a cell by administering a compound identified to a cell; and inhibiting retrovirus integration into the cell's genome. Also provided are pharmaceutical compositions comprising a compound identified by the screening methods and a pharmaceutically acceptable excipient. A compound that inhibits retroviral integration identified according to the methods herein disclosed. A compound that inhibits retroviral integration identified according to the methods of the invention wherein the compound is a lead compound for further development of a therapeutic agent that causes inhibition of retroviral integration into a host cell's genome.

Additionally provided are methods of inhibiting retroviral infectivity in a subject by administering the test compound identified to a subject and inhibiting retrovirus integration into the genome of the subject. In another embodiment are provided methods of screening for a compound which induces DNA repair in a cell wherein induction of DNA repair inhibits retroviral integration into a host cell's genome by exposing a component of a DNA repair pathway to a test compound; inducing DNA repair; measuring one of an amount of retroviral cDNA circle formation (via homologous recombination or non-homologous end-joining) by quantifying an expression of a marker gene, and the physical recombination of retroviral cDNA; inhibiting integration of the retroviral cDNA into the host cell genome; and identifying the compound. The component of the DNA repair pathway may be at least one of XPB or XPD, but not limited to the XPB or XPD members of the DNA repair pathway. Also encompassed by the invention are methods of inducing DNA repair in a cell wherein induction of DNA repair inhibits retroviral integration into the genome of the cell by administering a test compound identified by a method of the invention to a cell; inducing DNA repair; and inhibiting retrovirus integration into the genome of the cell. Other aspects of the invention include compounds that induce DNA repair identified according to a method of the invention wherein induction of DNA repair inhibits retroviral integration into a host cell's genome and pharmaceutical compositions of the compound and a pharmaceutically acceptable excipient.

One embodiment of the invention includes methods of inducing DNA repair in a subject by administering a test compound identified to a subject; inducing DNA repair; and inhibiting retrovirus integration into the subject's genome. The compound may induce DNA repair by upregulating a gene in a DNA repair pathway whereby DNA repair is induced and retroviral integration is inhibited or by inducing an activity or function of a protein in a DNA repair pathway whereby DNA repair is induced and retroviral integration is inhibited.

Also provided by the invention are methods of inducing DNA repair in a subject by administering a test compound identified by the methods of the invention to a subject and inducing DNA repair. Compounds that induce DNA repair identified according to methods of the invention may be lead compounds for further development of a therapeutic agent that causes inhibition of retroviral integration into a host cell's genome.

Another aspect of the invention includes methods of screening for a compound which induces DNA repair in a cell wherein induction of DNA repair inhibits retroviral integration into a host cell's genome by exposing a component of a DNA repair pathway to a test compound; inducing DNA repair; measuring one of an amount of retroviral cDNA circle formation (via homologous recombination or non-homologous end-joining) by quantifying an expression of a marker gene, and the physical recombination of retroviral cDNA; and identifying the compound.

The materials, methods, and examples provided herein are illustrative only and are not intended to be limiting. Other features and advantages of the invention will be apparent from the following detailed description and claims.

### BRIEF DESCRIPTION OF THE DRAWINGS

FIGS. 1A and 1B illustrate an example of retroviral infection of a host cell. FIG. 1A shows that HIV infection of a cell begins with the binding of the HIV envelope protein gp120 to the host cell membrane proteins CD4 and either CCR5 or CXCR4. This binding event elicits fusion of the retroviral and cellular membranes, mediated by a second HIV envelope protein gp41. Following membrane fusion, the retroviral capsid core enters the host cell and disassembles in the cytoplasm. HIV reverse transcriptase copies the retroviral genomic RNA into a cDNA molecule. The retroviral cDNA is part of the pre-integration complex (PIC), which includes at least the retroviral proteins integrase, reverse transcriptase, matrix, capsid, and vpr, as well as the host protein HMG I(Y). This complex of protein and nucleic acid enters the host cell nucleus. Retroviral integrase catalyzes the joining of the 3′ ends of the retroviral cDNA to the host genomic DNA. The retroviral cDNA is flanked by five base gaps of host sequence and 5′ dinucleotide flaps of HIV sequence. Host DNA repair enzymes finish the integration reaction by repairing the flanking gaps and 5′ flaps to generate the provirus. After integration is complete, retroviral and host transcription factors promote the transcription of retroviral mRNAs and genomic RNA. The retroviral mRNAs are translated in the cytoplasm to produce retroviral polyproteins. These polyproteins assemble at the cellular plasma membrane with the retroviral genomic RNA. Immature retroviral particles bud from the cell. After budding, the retroviral enzyme protease cleaves the retroviral polyproteins to yield a mature, infectious virion. FIG. 1B illustrates that, after the PIC enters the host cell nucleus, integration of the retroviral cDNA will result in a productive infection of the cell. Alternatively, circularization of the retroviral cDNA by one of at least two mechanisms is not productive and will prevent completion of retroviral infection. The host cellular DNA repair mechanism of homologous recombination may generate 1-long terminal repeat (1-LTR) circles. Both ends of the retroviral cDNA have homologous nucleotide sequences, termed long terminal repeats (LTRs). Host DNA repair machinery uses the homologous LTR ends in a recombination reaction to produce 1-LTR circles. A second host cellular DNA repair mechanism, non-homologous end-joining (NHEJ), ligates the ends of the retroviral cDNA to yield 2-long terminal repeat (2-LTR) circles. Neither 1-LTR nor 2-LTR circles can be subsequently converted to retroviral cDNA integration products.

FIG. 2 demonstrates that HIV cDNA integration is controlled by host cell DNA repair. HIV-based vector particles were used to determine relative retroviral cDNA integration efficiency in cell lines varying in DNA repair function. A successful retroviral cDNA integration event is indicated by the expression of green fluorescent protein (GFP) encoded by the HIV vector particles. Cell lines were derived from two patients with mutations of the XPB gene (Riou, L., L. Zeng, O. Chevallier-Lagente, A. Stary, O. Nikaido, A. Taieb, G. Weeda, M. Mezzina, and A. Sarasin. The relative expression of mutated XPB genes results in xeroderma pigmentosum/Cockayne's syndrome or trichothiodystrophy cellular phenotypes. Human Molecular Genetics, 8: 1125-1133, 1999). Three of the cell lines were rescued by addition of an XPB transgene. The five cell lines exhibit varying levels of DNA repair requiring XPB. The level of XPB function is indicated by triangles. The cell lines were transduced with the HIV-based vector particles at relative multiplicities of infection (MOI) of 0, 0.5, and 2, as determined by transduction of 293T human fibroblasts. Following transduction, the cells were fixed and examined by flow cytometry for GFP expression. Cells that did not have vector particles added, 0 MOI, did not express GFP. At both 0.5 MOI and 2 MOI, the percentage of cells expressing GFP (GFP+ cells) was inversely proportional to the level of XPB function.

FIGS. 3A and 3B illustrate one embodiment of a screen for retroviral cDNA circle-formation included within the scope of the invention. FIG. 3A shows a recombinant retroviral vector constructed to contain a general marker gene (for example, DsRed) driven by a promoter (for example, a cytomegalovirus (CMV) promoter or an MSH2 promoter). Detection of red fluorescence is used as a positive control for retroviral cDNA entry into the host cell nucleus. FIG. 3B illustrates that the formation of a 1-LTR or 2-LTR circle effectively juxtaposes a second promoter (for example, a CMV promoter or an MSH2 promoter) and a circularization marker gene (for example, GFP) with an intervening LTR (1-LTR or 2-LTR) that is flanked by 5′ splice donor and 3′ splice acceptor sites. Transcription from this second promoter results in a spliced message that has removed the intervening LTR(s) and will express the circularization marker gene, for example, GFP, and thus be detected, in the case of GFP, as green fluorescence. Because GFP is expressed only upon retroviral cDNA circularization, the level of green fluorescence indicates the efficiency of retroviral cDNA circle-formation versus retroviral cDNA integration into the host cell genome.

FIGS. 4A and 4B illustrate the nucleotide sequence of the human XPB gene (SEQ ID NO:1) and the amino acid sequence of the XPB polypeptide encoded thereby (SEQ ID NO:2), respectively (GenBank Accession No. NM_{—}000122). FIGS. 4C and 4D provide the nucleotide sequence of the human XPD gene (SEQ ID NO:3) and the amino acid sequence of the XPD polypeptide encoded thereby (SEQ ID NO:4), respectively (GenBank Accession No. NM_{—}000400).

FIGS. 5A-5D illustrate the nucleotide sequence (SEQ ID NO:5) of one example of the retroviral vector shown in FIG. 3, wherein the general marker gene is DsRed, expression of which is controlled by a CMV promoter, and the circularization marker gene is GFP, the expression of which is driven by a CMV promoter upon retroviral cDNA circularization.

FIGS. 6A-6D illustrate the nucleotide sequence (SEQ ID NO:6) of another example of the retroviral vector shown in FIG. 3, wherein the general marker is DsRed, expression of which is controlled by an MSH2 promoter, and the circularization marker gene is GFP, the expression of which is driven by a CMV promoter upon retroviral cDNA circularization.

### DETAILED DESCRIPTION OF ILLUSTRATIVE EMBODIMENTS

The reference works, patents, patent applications, and scientific literature that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology are known to those of skill in the art (Ausubel et al., CURRENT PROTOCOLS IN MOLECULAR BIOLOGY, John Wiley & Sons, New York, 1998; Sambrook et al, MOLECULAR CLONING: A LABORATORY MANUAL, 2D ED., Cold Spring Harbor Laboratory Press, Plainview, N.Y., 1989; Kaufman et al., Eds., HANDBOOK OF MOLECULAR AND CELLULAR METHODS IN BIOLOGY AND MEDICINE, CRC Press, Boca Raton, 1995; McPherson, Ed., DIRECTED MUTAGENESIS: A PRACTICAL APPROACH, IRL Press, Oxford, 1991).

The present invention relates to the processes whereby retroviruses insert their genetic material into the genome of a eukaryotic host cell in order to carry out a productive infection. More specifically, the present invention relates to highly conserved proteins of the host cell that are required for efficient retroviral cDNA integration. These proteins represent novel targets for anti-retroviral drugs and for drugs for improved gene delivery by retroviruses. Provided herein, inter alia, are methods and assay systems that can be used to screen for anti-retroviral compounds and compounds that increase retroviral gene delivery as well as to compare and test similar retroviral assays and drugs in vivo and in vitro.

The phrase “DNA repair pathway” as used herein refers to any pathway of a host cell that facilitates repair of the host DNA including but not limited to homologous recombination and non-homologous end-joining. A “component of a DNA repair pathway” refers to any molecule, including but not limited to nucleic acid molecules and polypeptides, that participates in a DNA repair pathway of a host cell. Examples of components of a DNA repair pathway include, but are not limited to, XPA, XPB, XPC, XPE, XPF, XPG, RAD50, RAD52, RAD54, RAD57, RAD59, MSH2, CDC9, hRAD50, hRAD51, hRAD51B, hRAD51C, hRAD51D, hXRCC2, hXRCC3, XRCC4, ligase IV, hMRE11, XRS2 (NBS 1), DNA-PK, and Ku70/80 heterodimer, and equivalent homologs.

As used herein, the term “contacting” means bringing together, either directly or indirectly, a compound into physical proximity to a molecule of interest. Contacting may occur, for example, in any number of buffers, salts, solutions, or in a cell or cell extract.

As used herein, the term “antibody” is meant to refer to complete, intact antibodies, and Fab, Fab′, F(ab)2, and other fragments thereof. Complete, intact antibodies include monoclonal antibodies such as murine monoclonal antibodies, chimeric antibodies, and humanized antibodies.

As used herein, the term “binding” means the physical or chemical interaction between two proteins or compounds or associated proteins or compounds or combinations thereof. Binding includes ionic, non-ionic, Hydrogen bonds, Van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates. Binding may be detected in many different manners. As a non-limiting example, the physical binding interaction between two molecules can be detected using a labeled compound. Other methods of detecting binding are well-known to those of skill in the art.

As used herein, the term “complementary” refers to Watson-Crick basepairing between nucleotide units of a nucleic acid molecule.

As used herein, the phrase “stringent hybridization conditions” or “stringent conditions” refers to conditions under which an oligonucleotide will specifically hybridize to its target sequence. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the oligonucleotides complementary to the target sequence hybridize to the target sequence at equilibrium. Since the target sequences are generally present in excess, at Tm, 50% of the hybridizing oligonucleotides are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short oligonucleotides (e.g., 10 to 50 nucleotides) and at least about 60° C. for longer oligonucleotides. Stringent conditions may also be achieved with the addition of destabilizing agents, such as formamide.

The term “marker gene” or “reporter gene” refers to a gene encoding a product that, when expressed, confers a phenotype at the physical, morphologic, or biochemical level on a transformed cell that is easily identifiable, either directly or indirectly, by standard techniques and includes, but is not limited to, green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), and xanthine guanine phosphoribosyltransferase (XGPRT). As with many of the standard procedures associated with the practice of the invention, skilled artisans will be aware of additional sequences that can serve the function of a marker or reporter. Thus, this list is merely meant to show examples of what can be used and is not meant to limit the invention. The term “general marker” or “general marker gene” as used herein refers to a gene of the retroviral cDNA that is expressed upon integration of the retroviral cDNA into the host genome or upon retroviral cDNA circularization and thus serves as a positive control for retroviral cDNA entry into the host cell nucleus. The term “circularization marker gene” or “circularization marker” refers to a gene of the retroviral cDNA that is expressed only upon circularization of the retroviral cDNA.

As used herein, the term “promoter” refers to a regulatory element that regulates, controls, or drives expression of a nucleic acid molecule of interest and can be derived from sources such as from adenovirus, SV40, parvoviruses, vaccinia virus, cytomegalovirus, or mammalian genomic DNA. Examples of suitable promoters for mammals include, but are not limited to, CMV and MSH2 promoters. Suitable promoters that can be used in yeast include, but are not limited to, such constitutive promoters as 3-phosphoglycerate kinase and various other glycolytic enzyme gene promoters or such inducible promoters as the alcohol dehydrogenase 2 promoter or metallothionine promoter. Again, as with many of the standard procedures associated with the practice of the invention, skilled artisans will be aware of additional promoters that can serve the function of directing the expression of a marker or reporter. Thus, the list is merely meant to show examples of what can be used and is not meant to limit the invention.

The terms “polypeptide,” “peptide,” and “protein are used interchangeably herein.

As used herein, the term “amino acid” denotes a molecule containing both an amino group and a carboxyl group. In some preferred embodiments, the amino acids are α-, β-, γ- or δ-amino acids, including their stereoisomers and racemates. As used herein the term “L-amino acid” denotes an α-amino acid having the L configuration around the α-carbon, that is, a carboxylic acid of general formula CH(COOH)(NH2)-(side chain), having the L-configuration. The term “D-amino acid” similarly denotes a carboxylic acid of general formula CH(COOH)(NH2)-(side chain), having the D-configuration around the α-carbon. Side chains of L-amino acids include naturally occurring and non-naturally occurring moieties. Non-naturally occurring (i.e., unnatural) amino acid side chains are moieties that are used in place of naturally occurring amino acid side chains in, for example, amino acid analogs. Amino acid substituents may be attached through their carbonyl groups through the oxygen or carbonyl carbon thereof, or through their amino groups, or through functionalities residing on their side chain portions.

As used herein “polynucleotide” refers to a nucleic acid molecule and includes genomic DNA, cDNA, RNA, mRNA and the like.

As used herein “antisense oligonucleotide” refers to a nucleic acid molecule that is complementary to at least a portion of a target nucleotide sequence of interest and specifically hybridizes to the target nucleotide sequence under physiological conditions. The term “double stranded RNA” or “dsRNA” as used herein refers to a double stranded RNA molecule capable of RNA interference, including short interfering RNA (siRNA) (see for example, Bass, Nature, 411, 428-429 (2001); Elbashir et al., Nature, 411, 494-498 (2001)).

“Synthesized” as used herein refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

“Retroviral cDNA circularization” refers to circle formation, for example 1-LTR or 2-LTR circle formation, of retroviral cDNA.

“Retroviral cDNA integration” as used herein refers to incorporation of retroviral cDNA into a host cell genomic DNA.

“Retroviral infection” as used herein refers to the process by which retroviruses propagate within a host cell and includes the steps of reverse transcription of retroviral RNA to retroviral cDNA and integration of retroviral cDNA into the host genome. “Noncircularized retroviral cDNA” or “linear retroviral cDNA” as used herein refers to retroviral cDNA that is not circularized into, for example, a 1-LTR or 2-LTR circle. “Circularized retroviral cDNA” refers to retroviral cDNA that is incapable of integration into a host cell genome and is in the form of a circle, for example, a 1-LTR or 2-LTR circle.

As used herein, the terms “modulates” or “modifies” means an increase or decrease in the amount, quality, or effect of a particular activity or protein.

“Inhibitors,” “activators,” and “modulators” refer to any inhibitory or activating molecules identified using in vitro and in vivo assays for, e.g., agonists, antagonists, and their homologs, including fragments, variants, and mimetics, as defined herein, that exert substantially the same biological activity as the molecule. “Inhibitors” are compounds that reduce, decrease, block, prevent, delay activation, inactivate, desensitize, or downregulate the biological activity or expression of a molecule or pathway of interest, e.g., antagonists. “Inducers” or “activators” are compounds that increase, induce, stimulate, open, activate, facilitate, enhance activation, sensitize, or upregulate a molecule or pathway of interest, e.g., agonists. In some embodiments of the invention, the level of inhibition or upregulation of the expression or biological activity of a molecule or pathway of interest refers to a decrease (inhibition or downregulation) or increase (upregulation) of greater than about 50%, 60%, 70%, 75%, 80%, 85%, 90%, 91%, 92%, 93%, 94%, 95%, 96%, 97%, 98%, or 99%. The inhibition or upregulation may be direct, i.e., operate on the molecule or pathway of interest itself, or indirect, i.e., operate on a molecule or pathway that affects the molecule or pathway of interest.

“About” as used herein refers to +/−10% of the reference value.

As used herein, “homologous nucleotide sequence” or “homologous amino acid sequence,” or variations thereof, refers to sequences characterized by a homology, at the nucleotide level or amino acid level, of at least about 60%, more preferably at least about 70%, more preferably at least about 80%, more preferably at least about 90%, at least about 95%, and most preferably at least about 98% to a reference sequence, or portion or fragment thereof encoding or having a functional domain, for example but not limited to the nucleic acid sequence of SEQ ID NO:1 or SEQ ID NO:3, or a portion of SEQ ID NO:1 or SEQ ID NO:3 which encodes a functional domain of the encoded polypeptide, SEQ ID NO:2 or SEQ ID NO:4, or polypeptides having amino acid sequence SEQ ID NO:2 or SEQ ID NO:4, or fragments thereof having functional domains of the full-length polypeptide. Homologous nucleotide sequences include those sequences coding for homologs, including, for example, isoforms, species variants, allelic variants, and fragments of the protein of interest. Isoforms can be expressed in different tissues of the same organism as a result of, for example, alternative splicing of RNA. Alternatively, isoforms can be encoded by different genes. Homologous nucleotide sequences include nucleotide sequences encoding for a species variant of a protein. Homologous nucleotide sequences also include, but are not limited to, naturally occurring allelic variations and mutations of the nucleotide sequences set forth herein. Homologous amino acid sequences include those amino acid sequences which encode conservative amino acid substitutions in polypeptides having amino acid sequence of SEQ ID NO:2 or SEQ ID NO:4, as well as in polypeptides identified according to the methods of the invention. Percent homology is preferably determined by, for example, the Gap program (Wisconsin Sequence Analysis Package, Version 8 for Unix, Genetics Computer Group, University Research Park, Madison Wis.), using the default settings, which uses the algorithm of Smith and Waterman (Smith and Waterman, Adv. Appl. Math., 2: 482-489, 1981). Nucleic acid fragments of the invention have at least about 5, at least about 10, at least about 15, at least about 20, at least about 25, at least about 50, or at least about 100 nucleotides of the reference nucleotide sequence. Preferably the nucleic acid fragments of the invention encode a polypeptide having at least one biological property, or function, that is substantially similar to a biological property of the polypeptide encoded by the full-length nucleic acid sequence.

As is well known in the art, because of the degeneracy of the genetic code, there are numerous DNA and RNA molecules that can code for the same polypeptide as that encoded by a nucleotide sequence of interest. The present invention, therefore, contemplates those other DNA and RNA molecules which, on expression, encode a polypeptide encoded by the nucleic acid molecule of interest. DNA and RNA molecules other than those specifically disclosed herein characterized simply by a change in a codon for a particular amino acid, are within the scope of this invention.

It is to be understood that the present invention includes proteins homologous to, and having at least one biological property, or function, that is substantially similar to a biological property of a reference polypeptide. Preferably, the extent of the biological activity of the biological property is at least 10%, more preferably at least 20%, more preferably at least 30%, more preferably at least 40%, more preferably at least 50%, more preferably at least 60%, more preferably at least 70%, more preferably at least 80%, more preferably at least 90%, and most preferably 100% of the activity of the biological property of the reference polypeptide. Such proteins are also called variants. This definition is intended to encompass fragments, isoforms, natural allelic variants, and splice variants. These variant forms may result from, for example, alternative splicing or differential expression in different tissue of the same source organism. The variant forms may be characterized by, for example, amino acid insertion(s), deletion(s), or substitution(s). In this connection, a variant form having an amino acid sequence which has at least about 60%, at least about 70% sequence homology, at least about 80% sequence homology, preferably about 90% sequence homology, more preferably about 95% sequence homology, and most preferably about 98% sequence homology to the reference polypeptide, is included in the present invention. A preferred homologous polypeptide comprises at least one conservative amino acid substitution compared to the reference polypeptide. Amino acid “insertions”, “substitutions” or “deletions” are changes to or within an amino acid sequence. The variation allowed in a particular amino acid sequence may be experimentally determined by producing the peptide synthetically or by systematically making insertions, deletions, or substitutions of nucleotides in the nucleic acid sequence using recombinant DNA techniques. Polypeptide fragments of the invention comprise at least about 5, 10, 15, 20, 25, 30, 35, or 40 consecutive amino acids of the reference polypeptide. Preferred polypeptide fragments display antigenic properties unique to, or specific for, the reference polypeptide and its allelic and species homologs. Fragments of the invention having the desired biological and immunological properties can be prepared by any of the methods well known and routinely practiced in the art.

Alterations of the naturally occurring amino acid sequence can be accomplished by any of a number of known techniques. For example, mutations can be introduced into the polynucleotide encoding a polypeptide at particular locations by procedures well known to the skilled artisan, such as oligonucleotide-directed mutagenesis, which is described by U.S. Pat. Nos. 4,518,584 and 4,737,462.

Preferably, a polypeptide homolog of the present invention will exhibit substantially the biological activity of a naturally occurring reference polypeptide. By “exhibit substantially the biological activity of a naturally occurring polypeptide” is meant that variants within the scope of the invention can comprise conservatively substituted sequences, meaning that one or more amino acid residues of a polypeptide are replaced by different residues that do not alter the secondary and/or tertiary structure of the polypeptide. Such substitutions may include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making phenotypically silent amino acid exchanges are known in the art (Bowie et al., Science, 247: 1306-1310, 1990). Other polypeptide homologs which might retain substantially the biological activities of the reference polypeptide are those where amino acid substitutions have been made in areas outside functional regions of the protein.

A nucleotide and/or amino acid sequence of a nucleic acid molecule or polypeptide employed in the invention or of a compound identified by the screening method of the invention may be used to search a nucleotide and amino acid sequence databank for regions of similarity using Gapped BLAST (Altschul et al., Nuc. Acids Res., 25: 3389, 1997). Briefly, the BLAST algorithm, which stands for Basic Local Alignment Search Tool is suitable for determining sequence similarity (Altschul et al., J. Mol. Biol., 215: 403-410, 1990). Software or performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., J. Mol. Biol., 215: 403-410, 1990). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The BLAST algorithm parameters W, T and X determine the sensitivity and speed of the alignment. The BLAST program uses as defaults a word length (W) of 11, the BLOSUM62 scoring matrix (Henikoff et al., Proc. Natl. Acad. Sci. USA, 89: 10915-10919, 1992) alignments (B) of 50, expectation (E) of 10, M=5, N=4, and a comparison of both strands. The BLAST algorithm (Karlin et al., Proc. Natl. Acad. Sci. USA, 90: 5873-5787, 1993) and Gapped BLAST perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a gene or cDNA if the smallest sum probability in comparison of the test nucleic acid to the reference nucleic acid is less than about 1, preferably less than about 0.1, more preferably less than about 0.01, and most preferably less than about 0.001.

“Biological activity” as used herein refers to the level of a particular function (for example, enzymatic activity) of a molecule or pathway of interest in a biological system. “Wild-type biological activity” refers to the normal level of function of a molecule or pathway of interest. “Reduced biological activity” refers to a decreased level of function of a molecule or pathway of interest relative to a reference level of biological activity of that molecule or pathway. For example, reduced biological activity may refer to a decreased level of biological activity relative to the wild-type biological activity of a molecule or pathway of interest. “Increased biological activity” refers to an increased level of function of a molecule or pathway of interest relative to a reference level of biological activity of that molecule or pathway. For example, increased biological activity may refer to an increased level of biological activity relative to the wild-type biological activity of a molecule or pathway of interest.

As used herein, the term “isolated” nucleic acid molecule refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules.

The term “mimetic” as used herein refers to a compound that is sterically similar to one identified as an inducer of a host DNA repair pathway, provided that the molecule retains biological activity, i.e., induction of a host DNA repair pathway. Mimetics are structural and functional equivalents to the compounds identified by the present invention that induce a DNA repair pathway.

The terms “patient” and “subject” are used interchangeably herein and include, but are not limited to, avians, felines, canines, bovines, ovines, porcines, equines, rodents, simians, and humans. “Host cell” includes, for example, a mammalian cell, yeast cell, or plant cell. Mammalian cells of the invention include but are not limited to human and chicken cells (e.g., DT40 cells).

The term “treatment” as used herein refers to any indicia of success of prevention, treatment, or amelioration of a retroviral infection, or to any indicia of success of improvement of the efficiency of gene delivery in a gene therapy. Treatment of a retroviral infection includesss any objective or subjective parameter, such as, but not limited to, abatement, remission, reduction in the number of retroviral particles in a patient, reduction in the number or severity of symptoms or side effects, an increase in the tolerance of the patient to the infection, or slowing of the rate of degeneration or decline of the patient. Treatment of a retroviral infection also includes a prevention of the onset of symptoms in a patient that may be at increased risk of retroviral infection but does not yet experience or exhibit symptoms thereof.

“Improving efficiency of gene delivery in a gene therapy” refers to any indicia of success of increasing the integration of a gene of a retrovirus or retroviral vector into the host cell genome. “Gene therapy” refers to any treatment method which introduces a gene into a patient for therapeutic effect, for example but not limited to, upregulation or downregulation of an endogenous nucleic acid or polypeptide.

Retroviral cDNA Integration

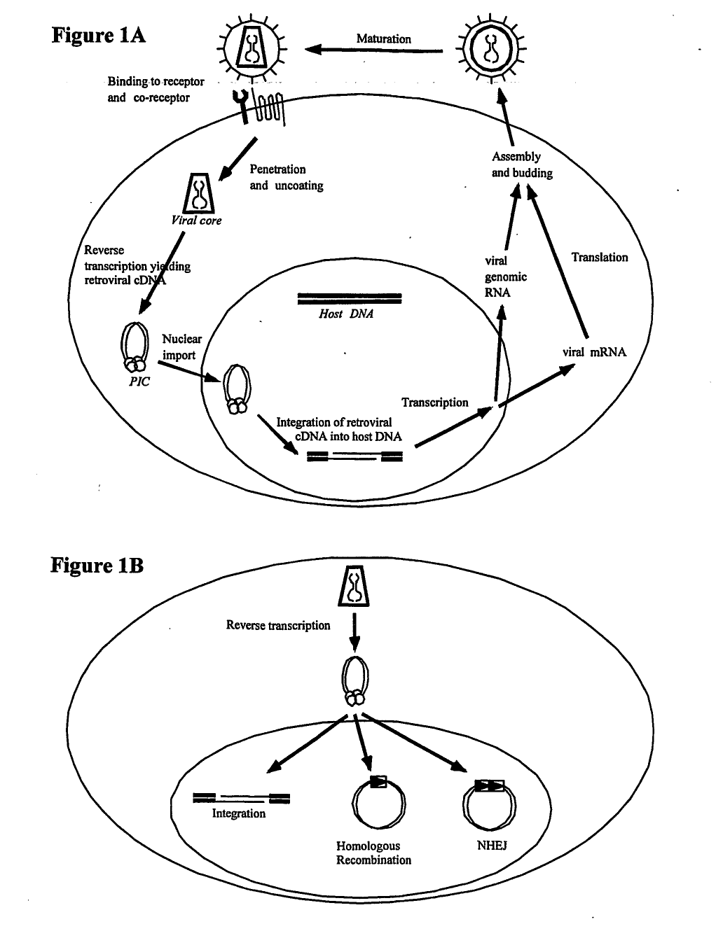

Some embodiments of the invention disclosed herein inhibit retroviral cDNA integration by stimulating a conserved cellular host defense mechanism, DNA repair. Other embodiments of the invention stimulate retroviral cDNA integration by inhibiting a conserved cellular host defense mechanism. Following reverse transcription, the retrovirus must integrate the cDNA copy of its genome into the host chromosome (Coffin, J. M., S. H. Hughes, and H. E. Varmus. RETROVIRUSES. Cold Spring Harbor, N.Y.: Cold Spring Harbor Laboratory Press, 1997; LaFemina, R. L., C. L. Schneider, H. L. Robbins, P. O. Callahan, K. LeGrow, E. Roth, W. A. Schleif, and E. A. E. Emini. Requirement of active human immunodeficiency virus type 1 integrase enzyme for productive infection of human T-lymphoid cells. Journal of Virology, 66: 7414-7419, 1992; Sakai, H., M. Kawamura, J. Sakuragi, S. Sakurgai, R. Shibata, A. Ishimoto, N. Ono, S. Ueda, and A. Adachi. Integration is essential for efficient gene expression of human immunodeficiency virus type 1. Journal of Virology, 67: 1169-1174, 1993; Englund, G., T. S. Theodore, E. O. Freed, A. Engleman, and M. A. Martin. Integration is required for productive infection of monocyte-derived macrophages by human immunodeficiency virus type 1. Journal of Virology, 69: 3216-3219, 1995). When integrated, the virus is termed a provirus. If a virus is unable to complete the formation of the integrated provirus, it will not be able to continue the infection. The process of retroviral cDNA integration, mediated by the pre-integration complex (PIC), is illustrated in FIG. 1A. Host factors that have been shown to influence the integration reaction include, but are not limited to, the high-mobility group protein family (HMGI(Y)), the barrier to autointegration factor (BAF), DNA-dependent protein kinase (DNA-PK), the Ku70/80 heterodimer, XRCC4, and ligase IV (Farnet, C. M., and F. D. Bushman. HIV-1 cDNA integration: requirement of HMG I(Y) protein for function of preintegration complexes in vitro. Cell, 88: 483-492, 1997; Lee, M. S., and R. Craigie. A previously unidentified host protein protects retroviral DNA from autointegration. Proceedings of the National Academy of Sciences, 95: 1528-1533, 1998; Daniel, R., R. A. Katz, A. M. Skalka. A role for DNA-PK in retroviral DNA integration. Science, 284, 1999; Li, L., J. M. Olvera, K. E. Yoder, R. S. Mitchell, S. L. Butler, M. Lieber, S. L. Martin, and F. D. Bushman. Role of the non-homologous DNA end-joining pathway in the early steps of retroviral infection. EMBO Journal, 20: 3272-3281, 2001). HMGI(Y) and BAF have both been shown to stimulate HIV retroviral cDNA integration in vitro. The proteins XRCC4, Ku70/80 heterodimer, and ligase IV catalyze non-homologous end joining (NHEJ) and are able to convert the linear retroviral cDNA to a circular molecule (2-LTR) joined at the long terminal repeat (LTR) sequences (FIG. 1B) (Li, L., J. M. Olvera, K. E. Yoder, R. S. Mitchell, S. L. Butler, M. Lieber, S. L. Martin, and F. D. Bushman. Role of the non-homologous DNA end-joining pathway in the early steps of retroviral infection. EMBO Journal, 20: 3272-3281, 2001). This 2-LTR circle form of retroviral cDNA is unable to integrate into the host cell genome (Brown, P. O., B. Bowerman, H. E. Varmus, and J. M. Bishop. Retroviral integration: structure of the initial covalent product and its precursor, and a role for the viral IN protein. Proceedings of the National Academy of Sciences, 86: 2525-2529, 1989; Engelman, A., G. Englund, J. M. Orenstein, M. A. Martin, and R. Craigie. Multiple effects of mutants in human immunodeficiency virus type 1 integrase on viral replication. Journal of Virology, 69: 2729-2736, 1995). An alternative fate for the linear retroviral cDNA is the formation of a 1-LTR circle formed by homologous recombination between the LTRs (FIG. 1B).

The results presented herein demonstrate that stimulation of the formation of 1-LTR and 2-LTR circles of the retroviral cDNA, for example by inducing a DNA repair pathway of a host cell, inhibits retroviral cDNA integration into the host genome and thus retroviral infectivity. Alternatively, inhibition of 1-LTR and/or 2-LTR circle formation of retroviral cDNA, for example, by inhibiting a DNA repair pathway, increases retroviral cDNA integration into a host cell genome and thus retroviral infectivity.

DNA Repair Genes Control the Efficiency of Integration

During a retroviral infection, nearly all of the linear viral cDNA will either integrate into the host genome or will become 1-LTR or 2-LTR circles (Zennou, V., C. Petit, D. Guetard, U. Nerhbass, L. Montagnier, and P. Charneau HIV-1 genomic nuclear import is mediated by a central DNA flap. Cell, 101: 173-185, 2000; Butler, S. L., M. S. T. Hansen, and F. D. Bushman. A quantitative assay for HIV DNA integration in vivo. Nature Medicine, 7: 631-634, 2001). Induction of host factors that mediate 1-LTR or 2-LTR circle formation increases the number of 1-LTR or 2-LTR circles, thereby resulting in a decrease in the number of integration events. Conversely, inhibition or knock-out of host factors that mediate 1-LTR or 2-LTR circle formation decreases retroviral cDNA circularization, thereby resulting in an increase in the number of integration events (Table 1). The invention presented herein describes strategies wherein linear retroviral cDNA molecules that are competent for integration are diverted to the alternative dead-end pathway of 1-LTR or 2-LTR circle formation. The invention also describes strategies for increasing the number of retroviral cDNA integration events by inhibiting 1-LTR or 2-LTR circle formation. Yeast studies suggest that the capacity of this system to control integration is quite large.

The yeast Saccharomyces cerevisiae has been shown to contain a retrovirus-like element family Ty (termed: retrotransposon). The Ty retrotransposon family contains the gag and pol genes indicative of retroviruses. The gag gene encodes all of the structural proteins associated with the virus-like particle. The pol gene includes reverse transcriptase, protease and integrase. Polyproteins are translated from the gag and pol genes and subsequently processed into functional proteins by the protease. Ty lacks an envelope (env) gene. Without an env gene, Ty particles are unable to bud from the yeast cell and therefore never exist outside the cell. Thus, Ty genomic RNA is transcribed and packaged in the cytoplasm as virus-like particles, that may then be uncoated, reverse transcribed, and integrated into the yeast genome. The lack of an extracellular stage of the life cycle is what defines Ty as a retrotransposon.

Studies of the Ty retrotransposon in yeast have shown that several yeast cellular DNA repair genes control the efficiency of retroviral cDNA integration. These repair genes include, but are not limited to, rad25, rad3, rad50, rad51, rad52, rad54, and rad57 (see, for example, Table 1; Lee, B.-S., C. P. Lichtenstein, B. Faiola, L. A. Rinckel, W. Wysock, M. J. Curcio, and D. J. Garfinkel. Posttranslational inhibition of Ty1 retrotransposition by nucleotide excision repair/transcription factor TFIIH subunits Ss12p and Rad3p. Genetics, 148: 1743-1761, 1998; Rattray, A. J., B. K. Shafer, and D. J. Garfinkel. The Saccharomyces cerevisiae DNA recombination and repair functions of the RAD52 epistasis group inhibit Ty1 transposition. Genetics, 154: 543-556, 2000). Mutation of these genes leads to great increases in integration efficiency. Conversely, the presence of wild-type DNA repair genes/proteins greatly reduces or prevents the integration reaction.

Three types of homologous recombination have been identified in eukaryotes that are distinguished by the amount of sequence homology required to induce recombination: microhomology recombination (requiring 1-5 base pairs of homologous sequence between participating parental DNA molecules), short-sequence recombination (requiring 20-300 base pairs of homologous sequence between participating parental DNA molecules), and homologous recombination (requiring >300 base pairs of homologous sequence between participating parental DNA molecules). Microhomology recombination appears to require Rad50p, MRE11p, XRS2(NBS1)p and a DNA ligase (presumed to be XRCC4/Lig4p). Short sequence and homologous recombination appear to require the rad52-pathway genes which include, but are not limited to: rad51, rad52, rad54, rad55, rad57, and rad59. In addition, the rad3 and rad25 genes also have been found to be part of the short-sequence homologous recombination pathway. All of these recombination pathway genes have human homologs, and all of the pathway types are conserved in human cells.

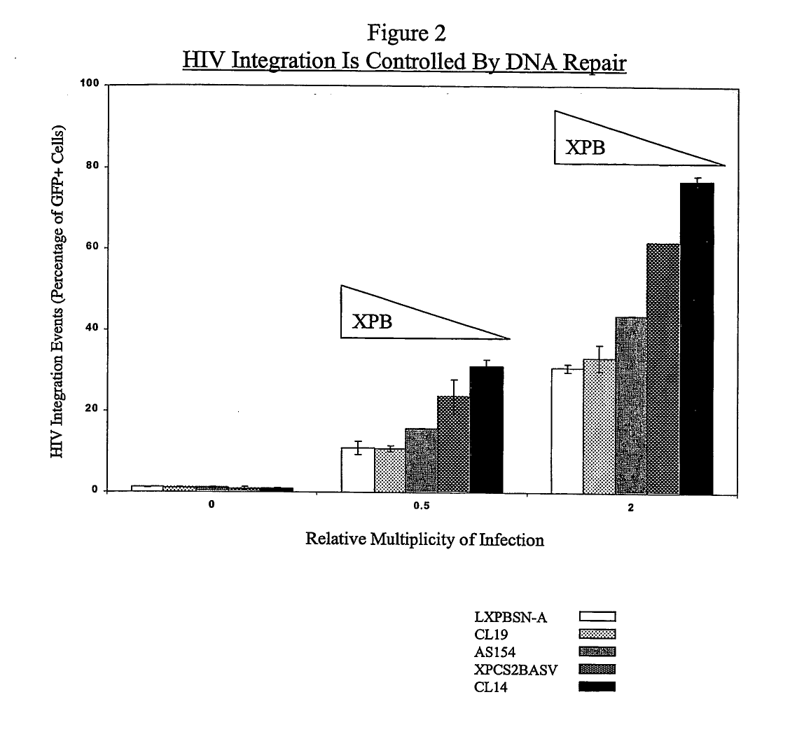

Indeed, the same host defense mechanism that inhibits or prevents Ty retrotransposon integration in the yeast S. cerevisiae is conserved in mammalian cells, including human. For example, human homologs of the yeast genes rad25, rad3, rad1, rad2, rad14, rad50, rad51, rad52, rad54, rad57, msh2, and cdc9 are XPB, XPD, XPF, XPG, XPA, hRAD50, hRAD51, hRAD52, hRAD54, hRAD57, hMSH2, and ligase I, respectively. A number of human genes, including but not limited to XPB, XPD, hRAD51, hMSH2, hRAD51B, hRAD51C, hRAD51D, hXRCC2, hXRCC3, have been identified as components of a human DNA repair pathway involving homologous recombination. The human homologs of Rad25p and Rad3p, XPB and XPD, respectively, inhibit integration of exogenous DNA (FIG. 2). XPB and XPD have been shown to be helicases that participate in two larger complexes of proteins: the transcription complex TFIIH and the nucleotide excision repair (NER) complex. In humans, mutations in at least one of the seven NER genes (XPA, XPB, XPC, XPD, XPE, XPF, and XPG) cause xeroderma pigmentosum (XP), a genetic disease associated with defective NER. NER factors work together and form multi-protein complexes on damaged DNA (Riou, L., L. Zeng, O. Chevallier-Lagente, A. Stary, O. Nikaido, A. Taieb, G. Weeda, M. Mezzina, and A. Sarasin. The relative expression of mutated XPB genes results in xeroderma pigmentosum/Cockayne's syndrome or trichothiodystrophy cellular phenotypes. Human Molecular Genetics, 8: 1125-1133, 1999).

The present-invention shows that-the DNA helicases XPB and XPD participate in the transformation of the linear retroviral cDNA to circularized retroviral cDNA, for example 1-LTR circles. The formation of 1-LTR circles is controlled by homologous recombination between the direct repeat LTRs of the retroviral cDNA. The level of retroviral cDNA integration inhibition is inversely proportional to the level of XPB repair activity in vivo (FIG. 2).

A second host cellular DNA repair mechanism, non-homologous end-joining (NHEJ), ligates the ends of the retroviral cDNA to yield 2-long terminal repeat (2-LTR) circles. The proteins DNA-PK, Ku70/80 heterodimer, XRCC4, ligase IV, hMRE11, hRAD50, and XRS2 (NBS1) participate in NHEJ. Members of the NHEJ pathway, including Ku70/80 heterodimer, ligase IV, and XRCC4, have been shown to convert the linear retroviral cDNA to a circular molecule (2-LTR) joined at the long terminal repeat (LTR) sequences (FIG. 1B).

DNA Repair Pathway and Anti-Retroviral Action

Inhibition of at least one component of a DNA repair pathway increases retroviral cDNA integration. Stimulation of at least one component of a DNA repair pathway decreases retroviral cDNA integration.

In some aspects of the present invention, genes and/or proteins within a DNA repair pathway are induced, that is, DNA repair is stimulated in order to inhibit retroviral cDNA integration. In some embodiments of the present invention the expression of a gene in a DNA repair pathway is upregulated, thereby increasing the production of at least one component of a DNA repair pathway. In some embodiments of the present invention, the biological activity or function of a protein involved in DNA repair is induced by a compound that interacts directly or indirectly with at least one component of a DNA repair pathway.

In some aspects of the present invention, genes and/or proteins within a DNA repair pathway are inhibited in order to increase retroviral cDNA integration. In some embodiments of the present invention the expression of a gene in a DNA repair pathway is down-regulated, thereby decreasing the production of at least one component of a DNA repair pathway. In some embodiments of the present invention, the activity or function of a protein involved in DNA repair is decreased by a compound that interacts directly or indirectly with at least one protein of a DNA repair pathway.

Screening for Compounds

The present invention provides methods for identifying compounds that modulate retroviral cDNA integration into a host genome. In some aspects of the invention, components of a DNA repair pathway have uses in the screening methods to detect molecules that specifically induce or inhibit components of a DNA repair pathway or bind the components of a DNA repair pathway to enhance or reduce their activity. In one embodiment, such assays are performed to screen molecules for utility as anti-retroviral drugs or lead compounds for drug development.

Methods of screening for compounds that modulate retroviral cDNA integration into the host genome include contacting a cell or cell extract with a non-circularized retroviral cDNA in the presence of a test compound and measuring the retroviral cDNA circularization that occurs. The amount of retroviral cDNA circularization that occurs in the presence of the test compound(s) may be compared with the retroviral cDNA circularization that occurs in comparable reaction medium that is not treated with the test compound(s). Compounds that increase retroviral cDNA integration cause a decrease of retroviral cDNA circularization as compared to the control in the absence of the test compound(s). Compounds that decrease retroviral cDNA integration cause an increase of retroviral cDNA circularization as compared to the control in the absence of the test compound(s).

Methods of screening for compounds that induce DNA repair include the steps of contacting one or more test compounds with one or more components of a DNA repair pathway of an organism of interest (which organism can be one of many different species, including, but not limited to, avians, felines, canines, bovines, ovines, porcines, equines, rodents, simians, and humans) in a suitable reaction medium and testing for compound/component interaction, e.g. by assessing the activity of the DNA repair pathway, or component thereof, and comparing that activity with the activity in comparable reaction medium that is not treated with the test compound(s). A difference in the activity between the treated and untreated samples is indicative of a modulating effect of the relevant test compound(s). Prior to being screened for the ability actually to affect or modulate DNA repair, test compounds may be screened for their ability to physically interact with a component of a DNA repair pathway. This may, for example, be used as a coarse screen prior to testing a compound for actual ability to modulate biological activity.

The components of a DNA repair pathway employed in the screening assay may be provided in a cell to be exposed to the test compound. Alternatively the assay may be performed on an in vitro DNA repair system that measures the accuracy and efficiency of joining together DNA strand breaks that have been created by treating intact DNA with restriction endonucleases, chemicals, radiation, or a recombinant retrovirus.

The activation of a DNA repair pathway leads to the protection of host DNA from degradation and thus protection from retroviral cDNA integration. Activation of a DNA repair pathway may be caused by DNA double-strand breaks (DSBs), single strand gaps in the DNA double helix, or by other disruptions to the DNA double-helix. These structures exist at the ends of retroviral cDNA and occur as intermediates in the retroviral cDNA integration process. Assays for DNA repair, retrovirus or retroviral cDNA, intermediates in retroviral cDNA integration, or synthetic preparations of DNA that mimic any of these may be provided.

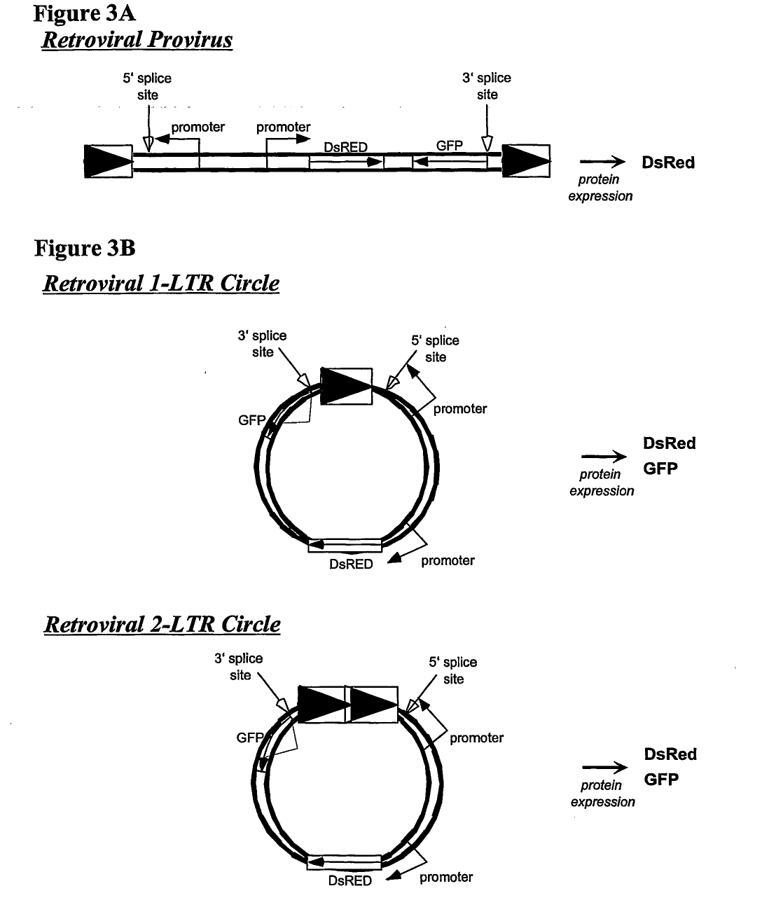

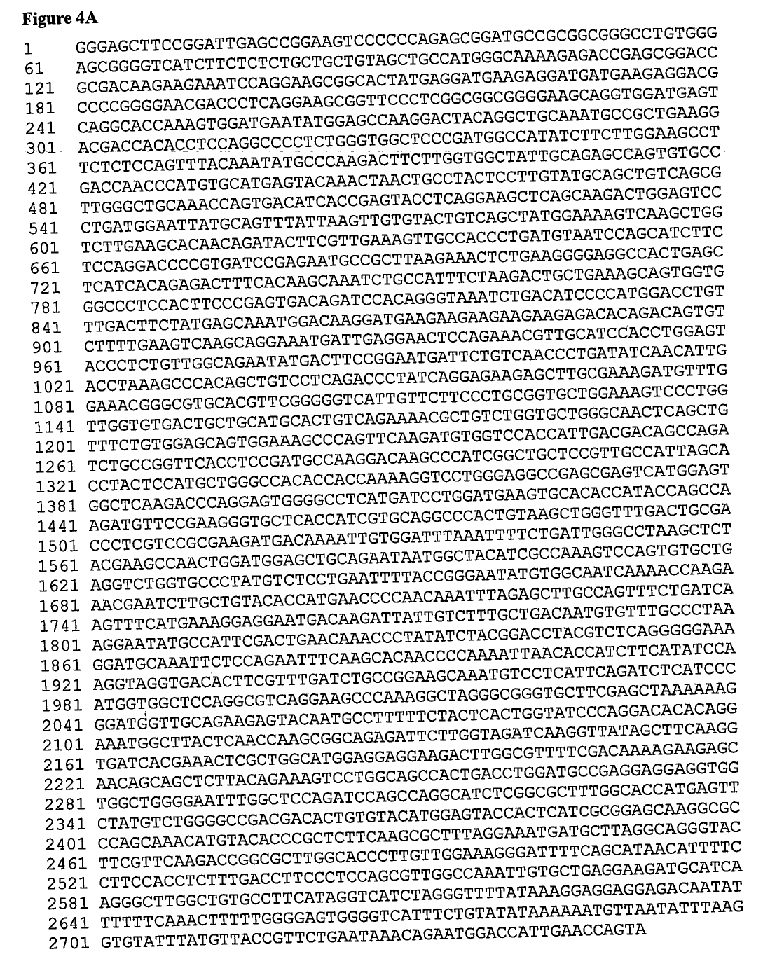

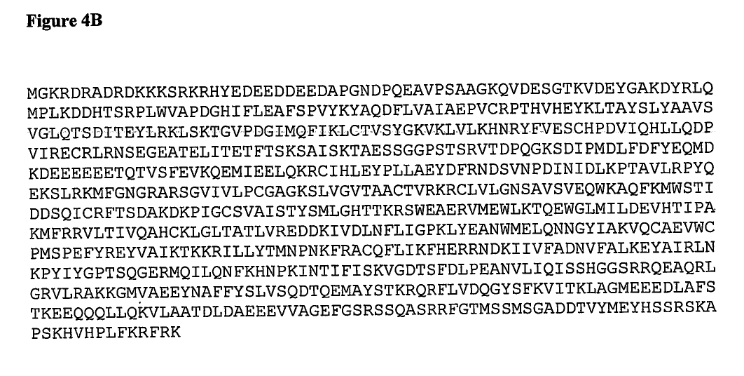

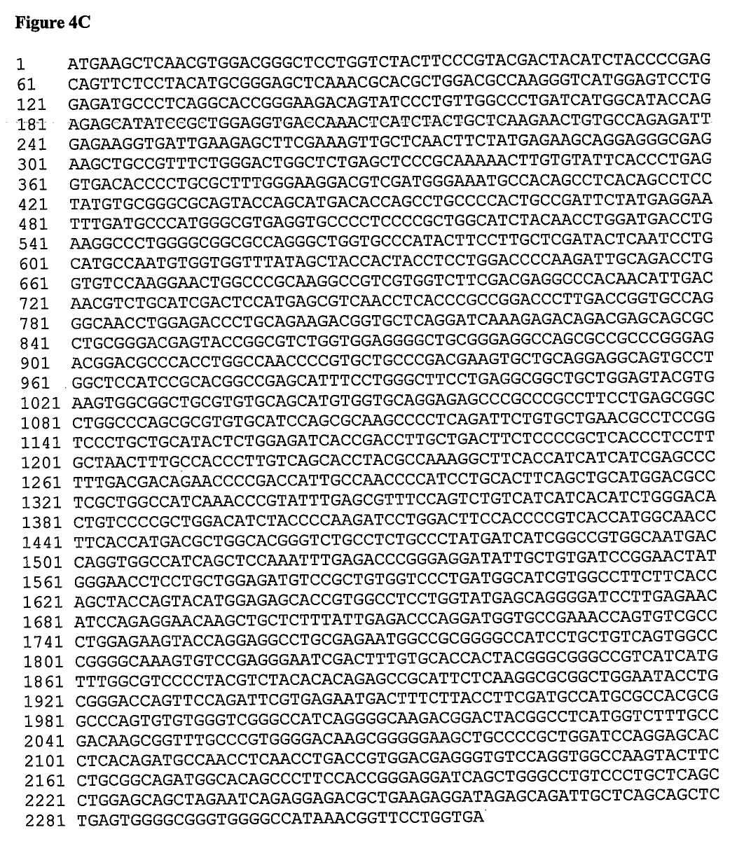

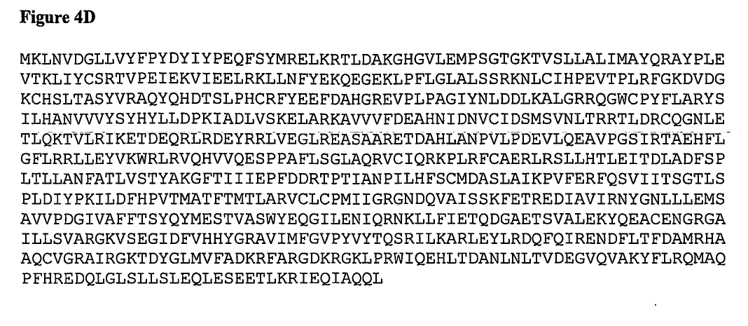

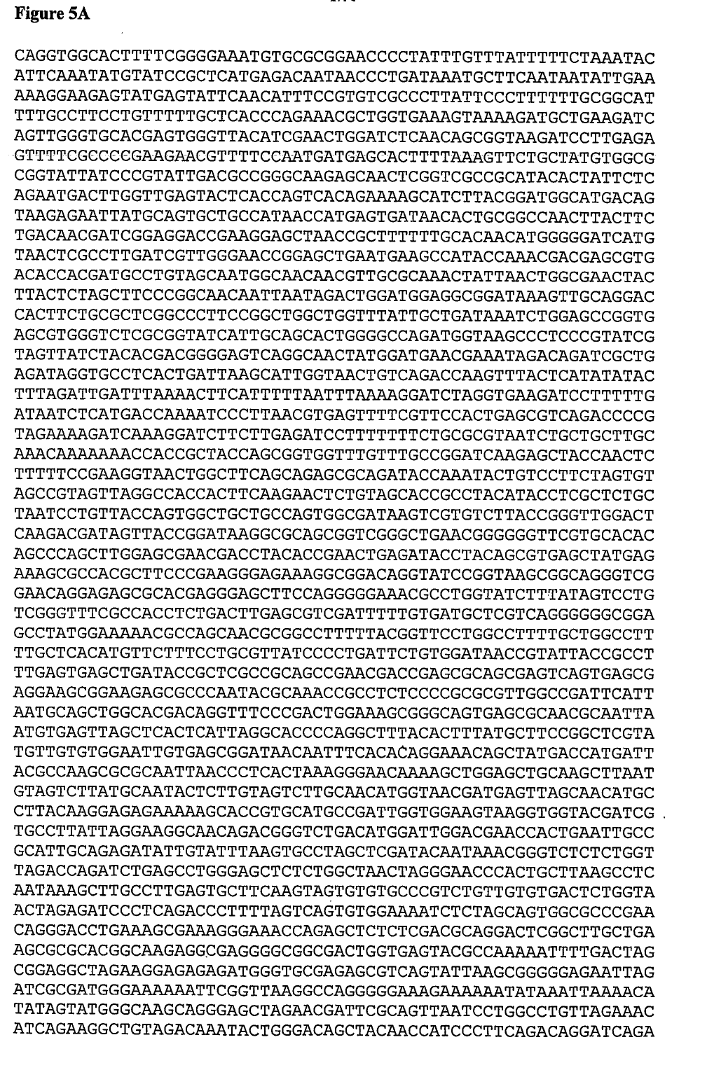

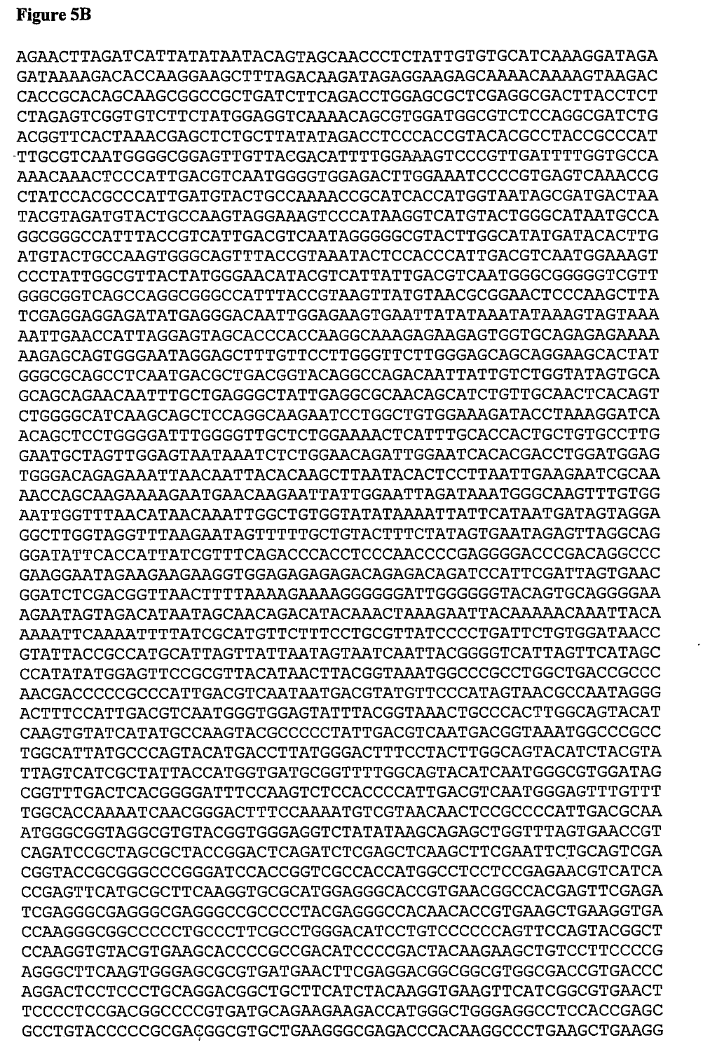

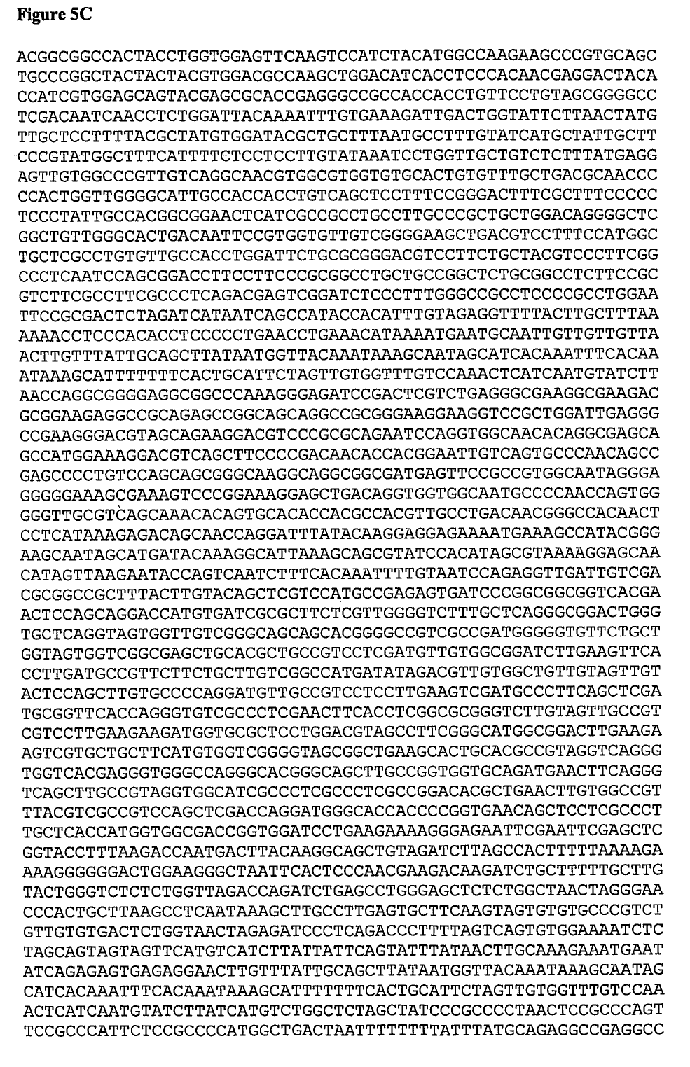

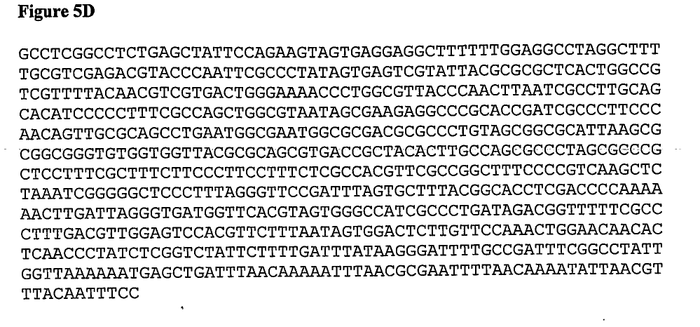

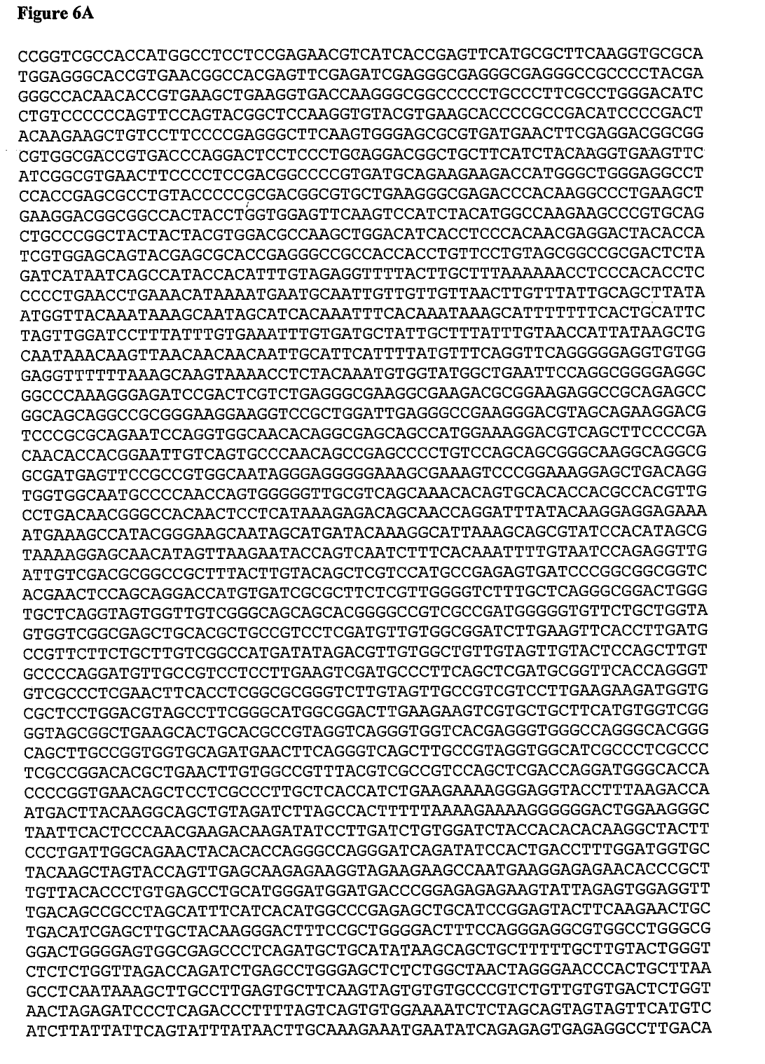

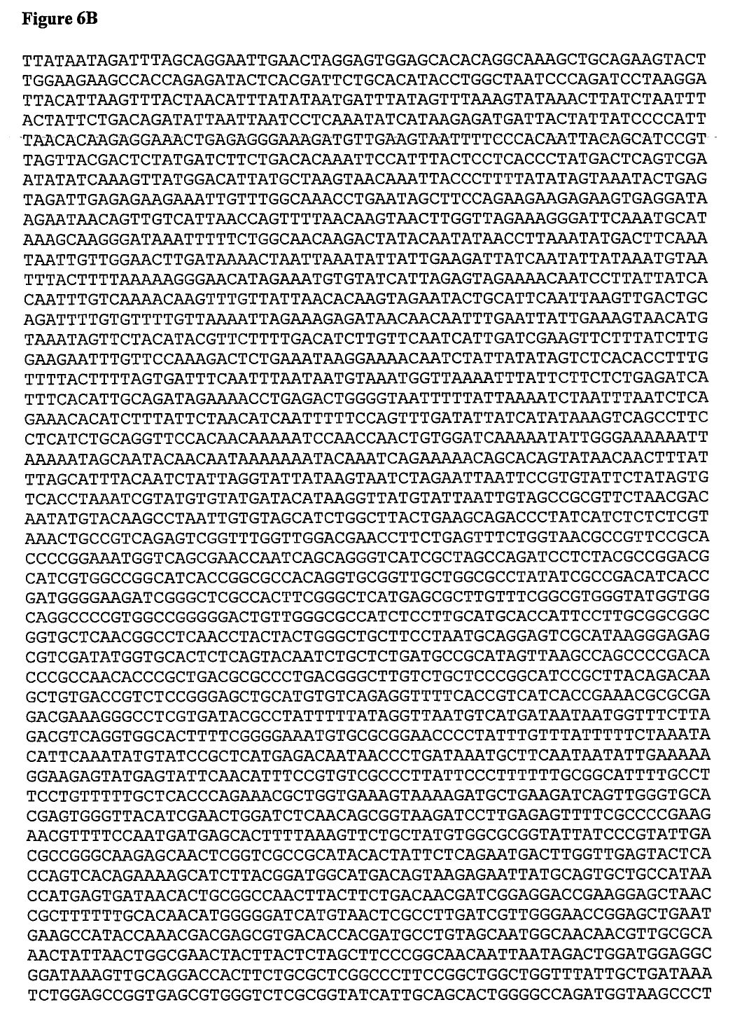

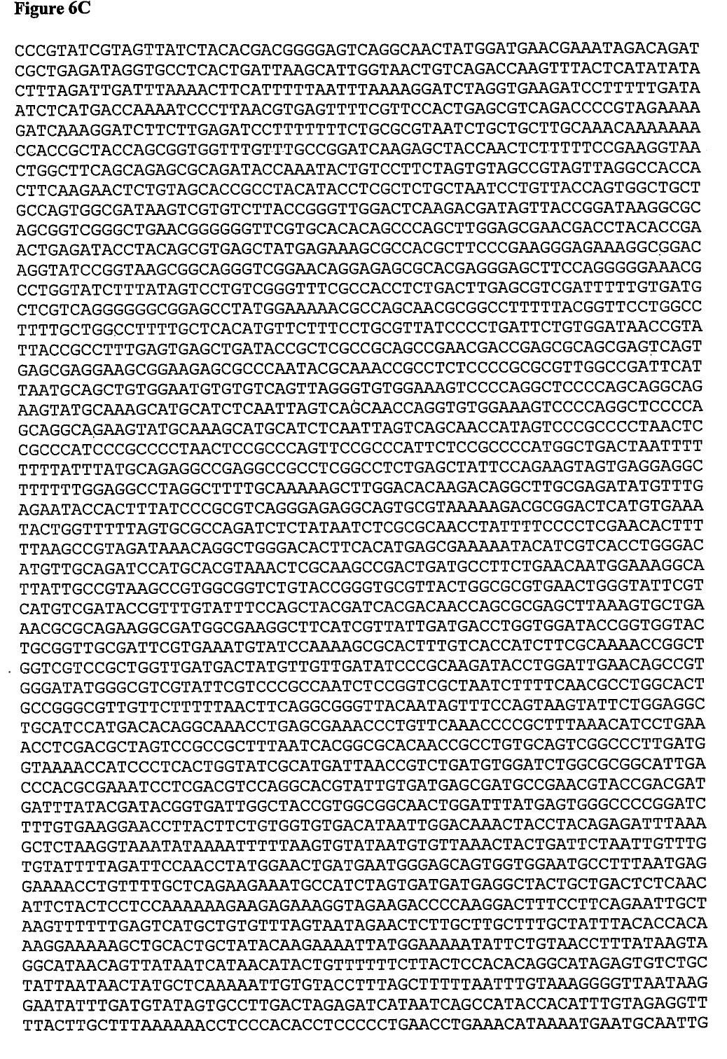

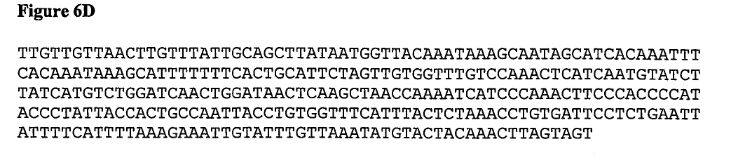

Methods of the invention identify compounds that modulate DNA repair and/or retroviral cDNA integration by their ability to modulate retroviral cDNA circle (1-LTR or 2-LTR) formation. Induction of DNA repair or inhibition of retroviral cDNA integration by the test compound is verified by an, increase in retroviral cDNA circle-formation. Inhibition of DNA repair or stimulation of retroviral cDNA integration by the test compound is verified by a decrease in retroviral cDNA circle-formation. Retroviral cDNA circle-formation is scored using standard genetic, biochemical, cellular, or histological techniques. For example, but not meant to limit the invention, a retroviral vector is designed such that the short-sequence homologous recombination that leads to the formation of the 1-LTR circles or non-homologous end-joining that leads to the formation of 2-LTR circles results in the juxtaposition of a promoter and a circularization marker gene, such as, but not limited to, green fluorescent protein (GFP) (FIG. 3). Proximity of the promoter to the marker gene results in expression of the marker gene, such as GFP, thereby allowing for the direct measurement of the expressed marker gene by cellular or biochemical techniques. The present invention also contemplates assaying for the ability of a test compound to affect the biological activity of a component of a DNA repair pathway. Thus, for example, compounds may be screened for their ability to affect DNA-PK phosphorylation, etc.

Screening of organic or peptide libraries with expressed recombinant protein components of a DNA repair pathway is useful for identification of therapeutic molecules that modulate the activity of a DNA repair pathway. In one embodiment screening is carried out to select for compounds that stimulate DNA repair as determined by the induction of 1-LTR or 2-LTR formation. In another embodiment, screening is performed to select for compounds that inhibit DNA repair as determined by the inhibition of 1-LTR or 2-LTR formation.

Diversity libraries, such as random or combinatorial peptide or non-peptide libraries are also screened for molecules that specifically stimulate or inhibit DNA repair. Many libraries are known in the art that can be used, such as, but not limited to, chemically synthesized libraries, recombinant (e.g., phage display libraries), and in vitro translation-based libraries. By way of examples of non-peptide libraries, a benzodiazepine library can be used. Peptide libraries can also be used. Another example of a library that can be used is one in which the amide functionalities in peptides have been permethylated to generate a chemically transformed combinatorial library. These methods are well known to those of skill in the art and can be found in standard molecular technique references.

Screening the libraries can be accomplished by any of a variety of commonly known methods.

The Test System

Host cells for the methods of the invention are preferably eukaryotic cells. Given the ease of manipulation of yeast, an assay according to the present invention may involve applying test compounds to a yeast system. Mammalian cells, including but not limited to human cells and chicken cells (e.g., DT40 cells), and plant cells also may be used in the methods of the invention.

For therapeutic purposes, a DNA repair pathway, or one or more components (or subunits) thereof, may be employed in the assay. The DNA repair pathway, or components thereof, may be, for example but not limited to, avian, feline, bovine, ovine, porcine, equine, rodent, simian, or human. In view of the high conservation between DNA repair components in different eukaryotes, similar results will be obtained using the compounds in mammalian, e.g. human, systems. In other words, a compound identified as being able to induce DNA repair in yeast will be able to induce DNA repair in other eukaryotes. A further approach is to employ standard recombinant technology techniques to generate yeast cells that express one or more components or subunits of a DNA repair pathway of another eukaryote, e.g. human. A plant DNA repair pathway, or one or more components thereof or cells comprising the components, may also be used in an assay according to the present invention to test for a compound(s) useful in modulating retrotransposon or retroelement activity in plants.

Alternatively, the system for screening for compounds in the methods of the invention may be cell-free, e.g., in a cell extract.

Compounds Identified by the Screening Methods

A compound that tests positive in an assay according to the present invention, i.e., is found to inhibit retroviral cDNA integration and/or stimulate DNA repair or, alternatively, is found to inhibit DNA repair and/or increase retroviral cDNA integration, may be peptide or non-peptide in nature. As used herein, the term “compound” means any identifiable chemical or molecule, including, but not limited to, small molecule, peptide, protein, sugar, nucleotide, or nucleic acid, and such compound can be natural or synthetic. Such compounds may include, for example, antibodies, antisense oligonucleotides, and small molecules. A “compound” identified by a screening method of the invention includes the compound so identified, in addition to homologs and mimetics thereof having the same functional effect on DNA repair and/or retroviral cDNA integration.

Antisense and siRNA

Compounds that inhibit DNA repair identified according to the methods of the invention include antisense oligonucleotides and small interfering RNA (siRNA) molecules to a component of a DNA repair pathway.

Antisense oligonucleotides are administered to cells or cell extract to disrupt at least one component of a DNA repair pathway. The antisense oligonucleotides hybridize to polynucleotides encoding a component of a DNA repair pathway. Both full-length and polynucleotide fragments are suitable for use as antisense oligonucleotides. “Antisense oligonucleotide fragments” of the invention include, but are not limited to oligonuclotides that specifically hybridize to DNA or RNA encoding a component of a DNA repair pathway (as determined by a sequence comparison of oligonucleotides encoding a component of a DNA repair pathway to oligonucleotides encoding other known polypeptides). Examples of antisense oligonucleotides of the invention include but are not limited to antisense oligonucleotides that hybridize to SEQ ID NO:1 or SEQ ID NO:3. Identification of sequences that are substantially unique to DNA repair component-encoding oligonucleotides can be ascertained by analysis of any publicly available sequence database and/or with any commercially available sequence comparison programs. Antisense molecules may be generated by any means including, but not limited to chemical synthesis, expression in an in vitro transcription reaction, expression in a transformed cell comprising a vector that may be transcribed to produce antisense molecules, restriction digestion and isolation, the polymerase chain reaction, and the like.

Those of skill in the art recognize that the antisense oligonucleotides that inhibit the expression and/or biological activity of a component of a DNA repair pathway may be predicted using any genes encoding a component of a DNA repair pathway. Specifically, antisense nucleic acid molecules comprise a sequence complementary to at least about 5, 10, 15, 20, 25, 30, 35, 40, 45, 50, 100, 250 or 500 nucleotides or an entire DNA repair gene sequence. Preferably, the antisense oligonucleotides comprise a sequence complementary to about 15 consecutive nucleotides of the coding strand of the DNA repair component-encoding sequence.

In one embodiment, an antisense nucleic acid molecule is antisense to a “coding region” of the coding strand of a nucleotide sequence encoding a DNA repair pathway component protein. The coding strand may also include regulatory regions of the DNA repair pathway component sequence. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding a DNA repair protein. The term “noncoding region” refers to 5′ and 3′ sequences which flank the coding region that are not translated into amino acids (i.e., also referred to as 5′ and 3′ untranslated regions (UTR)).

Antisense oligonucleotides may be directed to regulatory regions of a nucleotide sequence encoding a DNA repair protein, or mRNA corresponding thereto, including, but not limited to, the initiation codon, TATA box, enhancer sequences, and the like. Given the coding strand sequences provided herein, antisense nucleic acids of the invention can be designed according to the rules of Watson and Crick or Hoogsteen base pairing. The antisense nucleic acid molecule can be complementary to the entire coding region of a DNA repair component mRNA, but also may be an oligonucleotide that is antisense to only a portion of the coding or noncoding region of the mRNA. For example, the antisense oligonucleotide can be complementary to the region surrounding the translation start site of an mRNA. An antisense oligonucleotide can be, for example, about 5, 10, 15, 20, 25, 30, 35, 40, 45 or 50 nucleotides in length.

Another means to inhibit the activity of a DNA repair pathway component according to the invention is via RNA interference (RNAi) (see e.g., Elbashir et al., Nature, 411: 494-498 (2001); Elbashir et al., Genes Development, 15: 188-200 (2001)). RNAi is the process of sequence-specific, post-transcriptional gene silencing, initiated by double-stranded RNA (dsRNA) that is homologous in sequence to the silenced gene (e.g., is homologous in sequence to the sequence of a DNA repair pathway component, for example but not limited to the sequence as set forth in SEQ ID NO:1 or SEQ ID NO:3). siRNA-mediated silencing is thought to occur post-transcriptionally and/or transcriptionally. For example, siRNA duplexes may mediate post-transcriptional gene silencing by reconstitution of siRNA-protein complexes (siRNPs), which guide mRNA recognition and targeted cleavage.

Accordingly, another form of a DNA repair pathway inhibitory compound of the invention is a short interfering RNA (siRNA) directed against a DNA repair pathway component-encoding sequence. Exemplary siRNAs are siRNA duplexes (for example, 10-25, preferably 20, 21, 22, 23, 24 or 25 residues in length) having a sequence homologous or identical to a fragment of the XPB sequence set forth as SEQ ID NO:1 or the XPD sequence of SEQ ID NO:3, and having a symmetric 2-nucleotide 3′-overhang. The 2-nucleotide 3′ overhang is preferably composed of (2′-deoxy) thymidine because it reduces costs of RNA synthesis and may enhance nuclease resistance of siRNAs in the cell culture medium and within transfected cells. Substitution of uridine by thymidine in the 3′ overhang is also well tolerated in mammalian cells, and the sequence of the overhang appears not to contribute to target recognition.

Antibodies

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR) grafted antibodies, including compounds which include CDR sequences which specifically recognize a polypeptide of the invention) specific for components of a DNA repair pathway or fragments thereof. Preferred antibodies of the invention are human antibodies that are produced and identified according to methods described in WO93/11236, published Jun. 20, 1993. Antibody fragments, including Fab, Fab′, F(ab′)2, and Fv, are also provided by the invention. The term “specific for,” when used to describe antibodies of the invention, indicates that the variable regions of the antibodies of the invention recognize and bind a component of a DNA repair pathway exclusively (i.e., are able to distinguish the component from other known molecules by virtue of measurable differences in binding affinity, despite the possible existence of localized sequence identity, homology, or similarity). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds.), ANTIBODIES A LABORATORY MANUAL; Cold Spring Harbor Laboratory; Cold Spring Harbor, N.Y. (1988), Chapter 6. Antibodies that recognize and bind fragments of a component of a DNA repair pathway of the invention are also contemplated, provided that the antibodies are specific for the component of the DNA repair pathway. Antibodies of the invention can be produced using any method well known and routinely practiced in the art.

The invention provides an antibody that is specific for a component of a DNA repair pathway or an epitope thereof. Examples of antibodies of the invention include but are not limited to antibodies to the amino acid sequence of SEQ ID NO: 2 or SEQ ID NO:4, or epitopes thereof. Antibody specificity is described in greater detail below. Cross-reactive antibodies are not antibodies that are “specific” for a component of a DNA repair pathway. The determination of whether an antibody is specific or is cross-reactive with another molecule is made using any of several assays, such as Western blotting assays, that are well known in the art.

In one preferred variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for a component of a DNA repair pathway. Antisera isolated from an animal is an exemplary composition, as is a composition comprising an antibody fraction of an antisera that has been resuspended in water or in another diluent, excipient, or carrier.

In still another related embodiment, the invention provides an anti-idiotypic antibody specific for an antibody that is specific for a component of a DNA repair pathway.

It is well known that antibodies contain relatively small antigen binding domains that can be isolated chemically or by recombinant techniques. Such domains are useful DNA repair pathway component-binding molecules themselves, and also may be reintroduced into human antibodies, or fused to toxins or other polypeptides. Thus, in still another embodiment, the invention provides a polypeptide comprising a fragment of a DNA repair pathway component-specific antibody, wherein the fragment and the polypeptide bind to the component of a DNA repair pathway. By way of non-limiting example, the invention provides polypeptides that are single-chain antibodies and CDR (complementary determining region)-grafted antibodies.

Non-human antibodies may be humanized by any of the methods known in the art. In one method, the non-human CDRs are inserted into a human antibody or consensus antibody framework sequence. Further changes can then be introduced into the antibody framework to modulate affinity or immunogenicity.

Antibodies of the invention are useful for, e.g., therapeutic purposes (by modulating activity of a component of a DNA repair pathway).

Mimetics

Mimetics or mimics of compounds identified herein (sterically similar compounds formulated to mimic the key portions of the structure) may be designed for pharmaceutical use. Mimetics may be used in the same manner as the compounds identified by the present invention that stimulate DNA repair and hence are also functional equivalents. The generation of a structural-functional equivalent may be achieved by the techniques of modeling and chemical design known to those of skill in the art. It will be understood that all such sterically similar constructs fall within the scope of the present invention.

The designing of mimetics to a known pharmaceutically active compound is a known approach to the development of pharmaceuticals based on a “lead” compound. This is desirable where the active compound is difficult or expensive to synthesize, or where it is unsuitable for a particular method of administration, e.g. peptides are unsuitable active agents for oral compositions as they tend to be quickly degraded by proteases in the alimentary canal. There are several steps commonly taken in the design of a mimetic from a compound that induces DNA repair. First, the particular parts of the compound that are critical and/or important in determining its DNA repair-inducing properties are determined. In the case of a polypeptide, this can be done by systematically varying the amino acid residues in the peptide, e.g. by substituting each residue in turn. Alanine scans of peptides are commonly used to refine such peptide motifs.

Once the active region of the compound has been identified, its structure is modeled according to its physical properties, e.g. stereochemistry, bonding, size and/or charge, using data from a range of sources, such as, but not limited to, spectroscopic techniques, X-ray diffraction data, and NMR. Computational analysis, similarity mapping (which models the charge and/or volume of the active region, rather than the bonding between atoms), and other techniques known to those of skill in the art can be used in this modeling process.

In a variant of this approach, the three-dimensional structure of the compound that induces DNA repair and the active region of the target component of a DNA repair pathway are modeled. This can be especially useful where either or both of these compounds change conformation on binding.

A template molecule is then selected onto which chemical groups that mimic the compound that induces DNA repair can be grafted. The template molecule and the chemical groups grafted onto it can conveniently be selected so that the mimetic is easy to synthesize, is pharmacologically acceptable, and does not degrade in vivo, while retaining the biological activity of the lead compound. Alternatively, where the mimetic is peptide-based, further stability can be achieved by cyclizing the peptide, thereby increasing its rigidity. The mimetic or mimetics found by this approach can then be screened by the methods of the present invention to see whether they have the ability to induce DNA repair. Further optimization or modification can then be carried out to arrive at one or more final mimetics for in vivo or clinical testing.

Pharmaceutical Compositions

Following identification of a compound that induces DNA repair and/inhibits retroviral cDNA integration or, alternatively, inhibits DNA repair and/or stimulates retroviral cDNA integration, the compound may be manufactured and/or used in preparation of a pharmaceutical composition. These are administered to patients, including, but are not limited to, avians, felines, canines, bovines, ovines, porcines, equines, rodents, simians, and humans.

Thus, the present invention extends, in various aspects, not only to compounds identified in accordance with the methods disclosed herein but also pharmaceutical compositions, drugs, or other compositions comprising such a compound; methods comprising administration of such a composition to a patient, e.g. for treatment (which includes prophylactic treatment) of a retroviral disorder or for improving the efficiency of gene delivery in a gene therapy; uses of such a compound in the manufacture of a composition for administration to a patient; and methods of making a composition comprising admixing such a compound with a pharmaceutically acceptable excipient, vehicle or carrier, and optionally other ingredients.

The pharmaceutical compositions of the invention comprise a therapeutically effective amount of a compound identified according to the methods disclosed herein, or a pharmaceutically acceptable salt thereof, and a pharmaceutically acceptable carrier or excipient.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Pharmaceutically acceptable carriers include but are not limited to saline, buffered saline, dextrose, water, glycerol, ethanol, and combinations thereof. The carrier and composition can be sterile. The formulation should suit the mode of administration.

The composition, if desired, can also contain minor amounts of wetting or emulsifying agents or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium-carbonate, etc.

In one embodiment, the composition is formulated in accordance with routine procedures as a pharmaceutical composition adapted for oral (e.g., tablets, granules, syrups) or non-oral (e.g., ointments, injections) administration to the subject. Various delivery systems are known and can be used to administer a compound that induces DNA repair and/or inhibits retroviral cDNA integration, e.g., encapsulation in liposomes, microparticles, microcapsules, expression by recombinant cells, receptor-mediated endocytosis, construction of a therapeutic nucleic acid as part of a retroviral or other vector, etc. Methods of introduction include but are not limited to intradermal, intramuscular, intraperitoneal, intravenous, subcutaneous, intranasal, topical, and oral routes.

The compounds of the invention may be administered by any convenient route, for example by infusion or bolus injection, by absorption through epithelial or mucocutaneous linings (e.g., oral mucosa, rectal and intestinal mucosa, etc.), and may be administered together with other biologically active agents, for example in HAART therapy. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery; topical application, e.g., in conjunction with a wound dressing after surgery; by injection; by means of a catheter; by means of a suppository; or by means of an implant, said implant being of a porous, non-porous, or gelatinous material, including membranes, such as sialastic membranes, or fibers.

The composition can be administered in unit dosage form and may be prepared by any of the methods well known in the pharmaceutical art, for example, as described in REMINGTON'S PHARMACEUTICAL SCIENCES (Mack Publishing Co., Easton, Pa.). The amount of the compound of the invention that induces DNA repair and/or inhibits retroviral cDNA integration or, alternatively, that inhibits DNA repair and/or increase retroviral cDNA integration that is effective in the treatment of a particular disorder or condition will depend on factors including but not limited to the chemical characteristics of the compounds employed, the route of administration, the age, body weight, and symptoms of a patient, the nature of the disorder or condition, and can be determined by standard clinical techniques. Typically therapy is initiated at low levels of the compound and is increased until the desired therapeutic effect is achieved. In addition, in vitro assays may optionally be employed to help identify optimal dosage ranges. Suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry-lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline.

Where the composition is administered by injection, an ampoule of sterile water for injection or saline can be provided so that the ingredients may be mixed prior to administration.

Treatment Methods

The invention provides methods of treatment of retroviral infections by administration to a subject or patient of an effective amount of a compound that induces DNA repair and/or inhibits retroviral cDNA integration into the host genome. In some aspects of the invention, the compounds or pharmaceutical compositions of the invention are administered to a patient having an increased risk of or having a retroviral infection. The patient may be, for example, avian, feline, canine, bovine, ovine, porcine, equine, rodent, simian, or human. The retroviral infection may be associated with at least one of acquired immune deficiency syndrome (AIDS), human immunodeficiency virus (HLV) infection, cancer, human adult T-cell leukemia, lymphoma, FIV, Type I diabetes, and multiple sclerosis.

The invention also provides methods of treatment, for example, by improving gene delivery, by administering to a patient or subject an effective amount of a compound that increases retroviral cDNA integration and/or inhibits DNA repair. The patient may be, for example, avian, feline, canine, bovine, ovine, porcine, equine, rodent, simian, or human.

Kits of Retroviruses Having a Circularization Marker Gene

A kit of the invention comprises a carrier means being compartmentalized to receive in close confinement one or more container means such as vials, tubes, and the like, each of the container means comprising an element to be used in the methods of the invention. For example, one of the container means may comprise the retrovirus or retroviral vector of the invention having a circularization marker gene. The kit may also have one or more conventional kit components, including, but not limited to, instructions, test tubes, Eppendorf™ tubes, labels, reagents helpful for quantification of marker gene expression, etc.

TABLE 1
DNA Repair Pathway Component Knockouts Increase Integration

| Saccharomyces | Fold-increase | | Fold-increase |
|---|---|---|---|
| cerevisiae | Ty | Human | HIV |
| gene | Transposition¹ | gene | Integration² |
| rad25 | 2-1125³ | XPB | 2.5 |
| rad3 | 17-41³ | XPD | 2 |
| rad1 | 1 | | |
| rad2 | 1 | XPA | 1 |
| rad50 | 12 | | |
| rad51 | 11 | | |
| rad52 | 24 | | |
| rad54 | 5 | | |
| rad57 | 21 | | |
| msh2 | 1 | hMSH2 | 1 |
| cdc9 | 38 | | |

¹Continuous transposition/integration throughout the growth and analysis of cells.
²Single-round infection-generated increase in integration.
³Significant Stain Background effects

### Additional References

1. Temin, H. M., E. Keshet, and S. K. Weller. Correlation of transient accumulation of linear unintegrated viral DNA and transient cell killing by avian leukosis and reticuloendotheliosis viruses. Cold Spring Harbor Symposium on Quantitative Biology, 44: 773-778, 1980.
2. Pang, S., Y. Koyanagi, S. Miles, C. Wiley, H. V. Vintners, and I. S. Y. Chen. High levels of unintegrated HIV-1 DNA in brain tissue of AIDS dementia patients. Science, 343: 85-89, 1990.
3. Bergeron, L., and J. Sodroski. Dissociation of unintegrated viral DNA accumulation from single-cell lysis induced by human immunodeficiency virus type 1. Journal of Virology, 66: 5777-5787, 1992.
4. Berg, D. E., and M. M. Howe. Mobile DNA. Washington, D.C.: American Society for Microbiology, 1989.
5. Curcio, M. J., A. M. Hedge, J. D. Boeke, and D. J. Garfinkel. Ty RNA levels determine the spectrum of retrotransposition events that activate gene expression in Saccharomyces cerevisiae. Molecular General Genetics, 220: 213-221, 1990.
6. Curcio, M. J., and D. J. Garfinkel. Single-step selection for Ty1 element retrotransposition. Proceedings of the National Academy of Sciences, 88: 936-940, 1991.
7. Slupianek, A., C. Schmutte, G. Tombline, M. Nieborowska-Skorska, G. Hoser, M. O. Nowicki, A. J. Pierce, R. Fishel, and T. Skorski. BCR/ABL regulates mammalian RecA homologs, resulting in drug resistance. Molecular Cell, 8: 795-806, 2001.

1. A method of screening for a compound which induces a DNA repair pathway of a cell, comprising:
a) contacting at least one component of a DNA repair pathway with a non-circularized retroviral cDNA in the presence of a test compound;
b) contacting said at least one component of a DNA repair pathway with a non-circularized retroviral cDNA in the absence of said test compound; and
c) determining whether the amount of retroviral cDNA circularization is increased in the presence of said test compound relative to the amount of retroviral cDNA circularization in the absence of said test compound.

2. The method according to claim 1, wherein said component contacted with the test compound is a nucleic acid molecule encoding a polypeptide selected from the group consisting of XPA, XPB, XPC, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

3. The method according to claim 2, wherein said nucleic acid molecule encodes XPB or XPD.

4. The method according to claim 3, wherein said XPB has an amino acid sequence of SEQ ID NO:2 and wherein said XPD has an amino acid sequence of SEQ ID NO:4.

5. The method according to claim 3, wherein said nucleic acid molecule encoding XPB has a nucleotide sequence of SEQ ID NO:1 and wherein said nucleic acid molecule encoding XPD has a nucleotide sequence of SEQ ID NO:3.

6. The method according to claim 1, wherein said component contacted with the test compound is a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

7. The method according to claim 6, wherein said polypeptide is XPB or XPD.

8. The method according to claim 7, wherein said XPB has an amino acid sequence of SEQ ID NO:2 and wherein said XPD has an amino acid sequence of SEQ ID NO:4.

9. The method according to claim 1, wherein at least one component of said DNA repair pathway in the absence of said test compound exhibits reduced biological activity relative to wild-type biological activity of said component.

10. The method according to claim 9, wherein said component exhibiting reduced biological activity is a nucleic acid molecule encoding a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

11. The method according to claim 10, wherein said nucleic acid molecule encodes XPB or XPD.

12. The method according to claim 11, wherein said XPB has an amino acid sequence of SEQ ID NO:2 and wherein said XPD has an amino acid sequence of SEQ ID NO:4.

13. The method according to claim 11, wherein said nucleic acid molecule encoding XPB has a nucleotide sequence of SEQ ID NO:1 and wherein said nucleic acid molecule encoding XPD has a nucleotide sequence of SEQ ID NO:3.

14. The method according to claim 9, wherein said component exhibiting reduced biological activity is a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

15. The method according to claim 14, wherein said polypeptide is XPB or XPD.

16. The method according to claim 15, wherein said XPB has an amino acid sequence of SEQ ID NO:2 and wherein said XPD has an amino acid sequence of SEQ ID NO:4.

17. The method according to claim 1, wherein said test compound directly or indirectly upregulates the expression of at least one component of a DNA repair pathway.

18. The method according to claim 17, wherein said upregulated component of a DNA repair pathway is a nucleic acid molecule encoding a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

19. The method according to claim 18, wherein said nucleic acid molecule encodes XPB or XPD.

20. The method according to claim 19, wherein said XPB has an amino acid sequence of SEQ ID NO:2 and wherein said XPD has an amino acid sequence of SEQ ID NO:4.

21. The method according to claim 19, wherein said nucleic acid molecule encoding XPB has a nucleotide sequence of SEQ ID NO:1 and wherein said nucleic acid molecule encoding XPD has a nucleotide sequence of SEQ ID NO:3.

22. The method according to claim 1, wherein said test compound directly or indirectly upregulates the biological activity of at least one component of a DNA repair pathway.

23. The method according to claim 22, wherein said upregulated component of a DNA repair pathway is a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

24. The method according to claim 22, wherein said polypeptide is XPB or XPD.

25. The method according to claim 24, wherein said XPB has an amino acid sequence of SEQ ID NO:2 and wherein said XPD has an amino acid sequence of SEQ ID NO:4.

26. The method according to claim 1, wherein said retroviral cDNA comprises at least one marker gene and at least one promoter and wherein said marker gene is expressed from said promoter upon retroviral cDNA circularization.

27. The method according to claim 26, wherein said increase in retroviral cDNA circularization is detected by an increase in the level of expression of said marker gene in the presence of said test compound relative to the level of expression of said marker gene in the absence of said test compound.

28. The method according to claim 26, wherein said increase in retroviral cDNA circularization is detected by an increase in the level of activity of the polypeptide expressed from said marker gene in the presence of said test compound relative to the level of activity of the polypeptide expressed from said marker gene in the absence of said test compound.

29. The method according to claim 27, wherein said marker gene encodes green fluorescent protein (GFP), red fluorescent protein (DsRed), alkaline phosphatase (AP), α-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT).

30. The method according to claim 26, wherein said promoter is an adenovirus promoter, an SV40 promoter, a parvovirus promoter, a vaccinia virus promoter, a cytomegalovirus promoter, an MSH2 promoter, or a mammalian genomic DNA promoter.

31. The method according to claim 26, wherein said promoter is a 3-phosphoglycerate kinase gene promoter, an alcohol dehydrogenase-2 promoter, or a metallothionine promoter.

32. The method according to claim 1, wherein steps (a) and (b) occur in a cell or in cell extract.

33. The method according to claim 32, wherein said cell is a mammalian or yeast cell.

34. The method according to claim 1, wherein said compound inhibits retroviral cDNA integration into the genome of a cell.

35. The method of claim 34, wherein said compound prevents retroviral infection.

36. A compound that induces a DNA repair pathway of a cell identified according to the method of claim 1.

37. A pharmaceutical composition for the treatment of a retroviral infection comprising a therapeutically effective amount of at least one compound identified according to the method of claim 1, or a pharmaceutically acceptable salt thereof, and a pharmaceutically acceptable excipient.

38. A method of inducing a DNA repair pathway of a cell comprising administering at least one compound identified according to the method of claim 1 to said cell.

39. The method according to claim 38, wherein said compound inhibits retroviral cDNA integration into the genome of said cell.

40. A method of treating a retroviral infection of a patient comprising administering at least one compound identified according to the method of claim 1 to said patient.

41. The method according to claim 40, wherein said patient is a mammal.

42. The method according to claim 41, wherein said mammal is avian, feline, canine, bovine, ovine, porcine, equine, rodent, simian, or human.

43. The method according to claim 42, wherein said mammal is a human.

44. The method according to claim 40, wherein said retroviral infection is associated with at least one condition selected from the group consisting of acquired immune deficiency syndrome (AIDS), human immunodeficiency virus (HIV) infection, cancer, human adult T-cell leukemia, lymphoma, feline immunodeficiency virus (FIV), Type I diabetes, and multiple sclerosis.

45. The method according to claim 45, wherein said retroviral infection is HIV infection or AIDS.

46. A kit for identifying a compound that induces a DNA repair pathway comprising a retrovirus or retroviral vector having a marker gene that is expressed upon retroviral cDNA circularization.

47. The kit according to claim 46, further comprising at least one conventional kit component.

48. Use of a compound identified according to the method of claim 1 in the manufacture of a pharmaceutical composition for the treatment of a retroviral infection.

49. A method of identifying a compound that inhibits retroviral cDNA integration into a host genome comprising:
a) contacting a first cell or cell extract with a non-circularized retroviral cDNA in the presence of a test compound;
b) contacting a second cell or cell extract with a non-circularized retroviral cDNA in the absence of said test compound, wherein said first and said second cell or cell extract are of the same cell type; and
c) determining whether the amount of retroviral cDNA circularization is increased in the presence of said test compound relative to the amount of retroviral cDNA circularization in the absence of said test compound.

50. The method according to claim 49, wherein said retroviral cDNA comprises at least one marker gene and at least one promoter and wherein said marker gene is expressed from said promoter upon retroviral cDNA circularization.

51. The method according to claim 50, wherein said increase in retroviral cDNA circularization is detected by an increase in the level of expression of said marker gene in the presence of said test compound relative to the level of expression of said marker gene in the absence of said test compound.

52. The method according to claim 50, wherein said increase in retroviral cDNA circularization is detected by an increase in the level of activity of the polypeptide expressed from said marker gene in the presence of said test compound relative to the level of activity of the polypeptide expressed from said marker gene in the absence of said test compound.

53. The method according to claim 50, wherein said marker gene encodes green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT).

54. The method according to claim 50, wherein said promoter is an adenovirus promoter, an SV40 promoter, a parvovirus promoter, a vaccinia virus promoter, a cytomegalovirus promoter, an MSH2 promoter, or a mammalian genomic DNA promoter.

55. The method according to claim 50, wherein said promoter is a 3-phosphoglycerate kinase gene promoter, an alcohol dehydrogenase-2 promoter, or a metallothionine promoter.

56. The method according to claim 49, wherein said cell type is mammalian or yeast.

57. A compound that inhibits retroviral cDNA integration into a host cell genome identified according to the method of claim 49.

58. A pharmaceutical composition for the treatment of a retroviral infection comprising a therapeutically effective amount of at least one compound identified according to the method of claim 49, or a pharmaceutically acceptable salt thereof, and a pharmaceutically acceptable excipient.

59. A method of inhibiting retroviral cDNA integration into a host cell genome by administering a compound identified according to the method of claim 49 to said cell.

60. A method of treating a retroviral infection of a patient comprising administering at least one compound identified according to the method of claim 49 to said patient.

61. The method according to claim 60, wherein said patient is a mammal.

62. The method according to claim 61, wherein said mammal is avian, feline, canine, bovine, ovine, porcine, equine, rodent, simian, or human.

63. The method according to claim 62, wherein said mammal is a human.

64. The method according to claim 60, wherein said retroviral infection is associated with at least one condition selected from the group consisting of acquired immune deficiency syndrome (AIDS), human immunodeficiency virus (HIV) infection, cancer, human adult T-cell leukemia, lymphoma, feline immunodeficiency virus (FIV), Type I diabetes, and multiple sclerosis.

65. The method according to claim 64, wherein said retroviral infection is HIV infection or AIDS.

66. Use of a compound identified according to the method of claim 49 in the manufacture of a pharmaceutical composition for the treatment of a retroviral infection.

67. A kit for identifying a compound that inhibits retroviral cDNA integration into a host genome comprising a retrovirus or retroviral vector having a marker gene that is expressed upon retroviral cDNA circularization.

68. The kit according to claim 67, further comprising at least one conventional kit component.

69. A retroviral vector comprising a nucleic acid molecule having promoter and a marker gene that is expressed upon circularization of said nucleic acid molecule.

70. The retroviral vector of claim 69, wherein said marker gene encodes green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT).

71. The retroviral vector of claim 69, wherein said promoter is an adenovirus promoter, an SV40 promoter, a parvovirus promoter, a vaccinia virus promoter, a cytomegalovirus promoter, an MSH2 promoter, or a mammalian genomic DNA promoter.

72. The retroviral vector of claim 69, wherein said promoter is a 3-phosphoglycerate kinase gene promoter, an alcohol dehydrogenase-2 promoter, or a metallothionine promoter.

73. The retroviral vector of claim 69 comprising the nucleotide sequence of SEQ ID NO:5 or SEQ ID NO:6.

74. A method of screening for a compound which induces a DNA repair pathway of a cell, comprising:
a) contacting at least one component of a DNA repair pathway with a non-circularized retroviral cDNA in the presence of a test compound; and
b) determining the amount of retroviral cDNA circularization.

75. A method of identifying a compound that inhibits retroviral cDNA integration into a host genome comprising:
a) contacting a cell or cell extract with a non-circularized retroviral cDNA in the presence of a test compound; and
b) determining the amount of retroviral cDNA circularization.

76. A method of screening for a compound which inhibits a DNA repair pathway of a cell, comprising:
a) contacting at least one component of a DNA repair pathway with a non-circularized retroviral cDNA in the presence of a test compound;
b) contacting said at least one component of a DNA repair pathway with a non-circularized retroviral cDNA in the absence of said test compound; and
c) determining whether the amount of retroviral cDNA circularization is decreased in the presence of said test compound relative to the amount of retroviral cDNA circularization in the absence of said test compound.

77. The method according to claim 76, wherein said component contacted with the test compound is a nucleic acid molecule encoding a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

78. The method according to claim 77, wherein said nucleic acid molecule encodes XPB or XPD.

79. The method according to claim 78, wherein said XPB has an amino acid sequence of SEQ ID NO:2 and wherein said XPD has an amino acid sequence of SEQ ID NO:4.

80. The method according to claim 78, wherein said nucleic acid molecule encoding XPB has a nucleotide sequence of SEQ ID NO:1 and wherein said nucleic acid molecule encoding XPD has a nucleotide sequence of SEQ ID NO:3.

81. The method according to claim 76, wherein said component contacted with the test compound is a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

82. The method according to claim 81, wherein said polypeptide is XPB or XPD.

83. The method according to claim 82, wherein said XPB has an amino acid sequence of SEQ ID NO:2 and wherein said XPD has an amino acid sequence of SEQ ID NO:4.

84. The method according to claim 76, wherein said test compound directly or indirectly downregulates the expression of at least one component of a DNA repair pathway.

85. The method according to claim 84, wherein said downregulated component of a DNA repair pathway is a nucleic acid molecule encoding a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

86. The method according to claim 85, wherein said nucleic acid molecule encodes XPB or XPD.

87. The method according to claim 86, wherein said XPB has an amino acid sequence of SEQ ID NO:2 and wherein said XPD has an amino acid sequence of SEQ ID NO:4.

88. The method according to claim 86, wherein said nucleic acid molecule encoding XPB has a nucleotide sequence of SEQ ID NO:1 and wherein said nucleic acid molecule encoding XPD has a nucleotide sequence of SEQ ID NO:3.

89. The method according to claim 76, wherein said test compound directly or indirectly downregulates the biological activity of at least one component of a DNA repair pathway.

90. The method according to claim 89, wherein said downregulated component of a DNA repair pathway is a polypeptide selected from the group consisting of XPA, XPB, XPC, XPD, XPE, XPF, XPG, RAD50, RAD52, RAD54, RAD57, RAD59, MSH2, CDC9, hRAD51, hRAD51B, hRAD51C, hRAD51D, hXRCC2, hXRCC3, XRCC4, ligase IV, hMRE11, XRS2 (NBS1), DNA-PK, and Ku70/80 heterodimer, and homologs thereof.

91. The method according to claim 89, wherein said polypeptide is XPB or XPD.

92. The method according to claim 91, wherein said XPB has an amino acid sequence of SEQ ID NO:2 and wherein said XPD has an amino acid sequence of SEQ ID NO:4.

93. The method according to claim 76, wherein said retroviral cDNA comprises at least one marker gene and at least one promoter and wherein said marker gene is expressed from said promoter upon retroviral cDNA circularization.

94. The method according to claim 93, wherein said decrease in retroviral cDNA circularization is detected by a decrease in the level of expression of said marker gene in the presence of said test compound relative to the level of expression of said marker gene in the absence of said test compound.

95. The method according to claim 93, wherein said decrease in retroviral cDNA circularization is detected by a decrease in the level of activity of the polypeptide expressed from said marker gene in the presence of said test compound relative to the level of activity of the polypeptide expressed from said marker gene in the absence of said test compound.

96. The method according to claim 93, wherein said marker gene encodes green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding β-galactosidase), luciferase (luc), or xanthine guanine phosphoribosyltransferase (XGPRT).

97. The method according to claim 93, wherein said promoter is an adenovirus promoter, an SV40 promoter, a parvovirus promoter, a vaccinia virus promoter, a cytomegalovirus promoter, an MSH2 promoter, or a mammalian genomic DNA promoter.

98. The method according to claim 93, wherein said promoter is a 3-phosphoglycerate kinase gene promoter, an alcohol dehydrogenase-2 promoter, or a metallothionine promoter.

99. The method according to claim 76, wherein steps (a) and (b) occur in a cell or in cell extract.

100. The method according to claim 99, wherein said cell is a mammalian or yeast cell.

101. The method according to claim 76, wherein said compound increases retroviral cDNA integration into the genome of a cell.

102. A compound that inhibits a DNA repair pathway of a cell identified according to the method of claim 76.

103. A pharmaceutical composition for increasing efficiency of gene delivery in a gene therapy comprising a therapeutically effective amount of at least one compound identified according to the method of claim 76, or a pharmaceutically acceptable salt thereof, and a pharmaceutically acceptable excipient.

104. A method of inhibiting a DNA repair pathway of a cell comprising administering at least one compound identified according to the method of claim 76 to said cell.

105. The method according to claim 104, wherein said compound increases retroviral cDNA integration into the genome of said cell.

106. A method of improving efficiency of gene delivery in a gene therapy of a patient comprising administering at least one compound identified according to the method of claim 76 to said patient.

107. The method according to claim 106, wherein said patient is a mammal.

108. The method according to claim 107, wherein said mammal is avian, feline, canine, bovine, ovine, porcine, equine, rodent, simian, or human.

109. The method according to claim 108, wherein said mammal is a human.

110. A kit for identifying a compound that inhibits a DNA repair pathway comprising a retrovirus or retroviral vector having a marker gene that is expressed upon retroviral cDNA circularization.

111. The kit according to claim 110, further comprising at least one conventional kit component.

112. Use of a compound identified according to the method of claim 76 in the manufacture of a pharmaceutical composition for increasing the efficiency of gene delivery in a gene therapy.

113. A method of identifying a compound that increases retroviral cDNA integration into a host genome comprising:
a) contacting a first cell or cell extract with a non-circularized retroviral cDNA in the presence of a test compound;
b) contacting a second cell or cell extract with a non-circularized retroviral cDNA in the absence of said test compound, wherein said first and said second cell or cell extract are of the same cell type; and
c) determining whether the amount of retroviral cDNA circularization is decreased in the presence of said test compound relative to the amount of retroviral cDNA circularization in the absence of said test compound.

114. The method according to claim 113, wherein said retroviral cDNA comprises at least one marker gene and at least one promoter and wherein said marker gene is expressed from said promoter upon retroviral cDNA circularization.

115. The method according to claim 113, wherein said decrease in retroviral cDNA circularization is detected by a decrease in the level of expression of said marker gene in the presence of said test compound relative to the level of expression of said marker gene in the absence of said test compound.

116. The method according to claim 114, wherein said decrease in retroviral cDNA circularization is detected by a decrease in the level of activity of the polypeptide expressed from said marker gene in the presence of said test compound relative to the level of activity of the polypeptide expressed from said marker gene in the absence of said test compound.

117. The method according to claim 114, wherein said marker gene encodes green fluorescent protein (GFP), red fluorescent protein (DsRed), alkaline phosphatase (AP), β-lactamase, chloramphenicol acetyltransferase (CAT), adenosine deaminase (ADA), aminoglycoside phosphotransferase (neor, G418r) dihydrofolate reductase-(DHFR), hygromycin-B-phosphotransferase (HPH), thymidine kinase (TK), lacZ (encoding β-galactosidase), luciferase (luc), or xanthine guanine phosphoribosyltransferase (XGPRT).

118. The method according to claim 114, wherein said promoter is an adenovirus promoter, an SV40 promoter, a parvovirus promoter, a vaccinia virus promoter, a cytomegalovirus promoter, an MSH2 promoter, or a mammalian genomic DNA promoter.

119. The method according to claim 114, wherein said promoter is a 3-phosphoglycerate kinase gene promoter, an alcohol dehydrogenase-2 promoter, or a metallothionine promoter.

120. The method according to claim 113, wherein said cell type is mammalian or yeast.

121. A compound that increases retroviral cDNA integration into a host cell genome identified according to the method of claim 113.

122. A pharmaceutical composition for the increasing the efficiency of gene delivery in a gene therapy comprising a therapeutically effective amount of at least one compound identified according to the method of claim 113, or a pharmaceutically acceptable salt thereof, and a pharmaceutically acceptable excipient.

123. A method of increasing retroviral cDNA integration into a host cell genome by administering a compound identified according to the method of claim 113 to said cell.

124. A method of improving the efficiency of gene delivery of a gene therapy of a patient comprising administering at least one compound identified according to the method of claim 113 to said patient.

125. The method according to claim 124, wherein said patient is a mammal.

126. The method according to claim 125, wherein said mammal is avian, feline, bovine, ovine, porcine, equine, rodent, simian, or human.

127. The method according to claim 126, wherein said mammal is a human.

128. Use of a compound identified according to the method of claim 113 in the manufacture of a pharmaceutical composition for improving the efficiency of gene delivery in a gene therapy.

129. A kit for identifying a compound that increases retroviral cDNA integration into a host genome comprising a retrovirus or retroviral vector having a marker gene that is expressed upon retroviral cDNA circularization.

130. The kit according to claim 129, further comprising at least one conventional kit component.

131. A method of screening for a compound which inhibits a DNA repair pathway of a cell, comprising:
a) contacting at least one component of a DNA repair pathway with a non-circularized retroviral cDNA in the presence of a test compound; and
b) determining the amount of retroviral cDNA circularization.

132. A method of identifying a compound that increases retroviral cDNA integration into a host genome comprising:
a) contacting a cell or cell extract with a non-circularized retroviral cDNA in the presence of a test compound; and
b) determining the amount of retroviral cDNA circularization.


SEQUENCE LISTING (6 sequences)

SEQ ID NO: 1 - DNA, 2751 bp, Homo sapiens
gggagcttccggattgagccggaagtccccccagagcggatgccgcggcgggcctgtgggagcggggtcatcttctctctgctgctgtagctgccatgggcaaaagagaccgagcggaccgcgacaagaagaaatccaggaagcggcactatgaggatgaagaggatgatgaagaggacgccccggggaacgaccctcaggaagcggttccctcggcggcggggaagcaggtggatgagtcaggcaccaaagtggatgaatatggagccaaggactacaggctgcaaatgccgctgaaggacgaccacacctccaggcccctctgggtggctcccgatggccatatcttcttggaagccttctctccagtttacaaatatgcccaagacttcttggtggctattgcagagccagtgtgccgaccaacccatgtgcatgagtacaaactaactgcctactccttgtatgcagctgtcagcgttgggctgcaaaccagtgacatcaccgagtacctcaggaagctcagcaagactggagtccctgatggaattatgcagtttattaagttgtgtactgtcagctatggaaaagtcaagctggtcttgaagcacaacagatacttcgttgaaagttgccaccctgatgtaatccagcatcttctccaggaccccgtgatccgagaatgccgcttaagaaactctgaaggggaggccactgagctcatcacagagactttcacaagcaaatctgccatttctaagactgctgaaagcagtggtgggccctccacttcccgagtgacagatccacagggtaaatctgacatccccatggacctgtttgacttctatgagcaaatggacaaggatgaagaagaagaagaagagacacagacagtgtcttttgaagtcaagcaggaaatgattgaggaactccagaaacgttgcatccacctggagtaccctctgttggcagaatatgacttccggaatgattctgtcaaccctgatatcaacattgacctaaagcccacagctgtcctcagaccctatcaggagaagagcttgcgaaagatgtttggaaacgggcgtgcacgttcgggggtcattgttcttccctgcggtgctggaaagtccctggttggtgtgactgctgcatgcactgtcagaaaacgctgtctggtgctgggcaactcagctgtttctgtggagcagtggaaagcccagttcaagatgtggtccaccattgacgacagccagatctgccggttcacctccgatgccaaggacaagcccatcggctgctccgttgccattagcacctactccatgctgggccacaccaccaaaaggtcctgggaggccgagcgagtcatggagtggctcaagacccaggagtggggcctcatgatcctggatgaagtgcacaccataccagccaagatgttccgaagggtgctcaccatcgtgcaggcccactgtaagctgggtttgactgcgaccctcgtccgcgaagatgacaaaattgtggatttaaattttctgattgggcctaagctctacgaagccaactggatggagctgcagaataatggctacatcgccaaagtccagtgtgctgaggtctggtgccctatgtctcctgaattttaccgggaatatgtggcaatcaaaaccaagaaacgaatcttgctgtacaccatgaaccccaacaaatttagagcttgccagtttctgatcaagtttcatgaaaggaggaatgacaagattattgtctttgctgacaatgtgtttgccctaaaggaatatgccattcgactgaacaaaccctatatctacggacctacgtctcagggggaaaggatgcaaattctccagaatttcaagcacaaccccaaaattaacaccatcttcatatccaaggtaggtgacacttcgtttgatctgccggaagcaaatgtcctcattcagatctcatcccatggtggctccaggcgtcaggaagcccaaaggctagggcgggtgcttcgagctaaaaaagggatggttgcagaagagtacaatgcctttttctactcactggtatcccaggacacacaggaaatggcttactcaaccaagcggcagagattcttggtagatcaaggttatagcttcaaggtgatcacgaaactcgctggcatggaggaggaagacttggcgttttcgacaaaagaagagcaacagcagctcttacagaaagtcctggcagccactgacctggatgccgaggaggaggtggtggctggggaatttggctccagatccagccaggcatctcggcgctttggcaccatgagttctatgtctggggccgacgacactgtgtacatggagtaccactcatcgcggagcaaggcgcccagcaaacatgtacacccgctcttcaagcgctttaggaaatgatgcttaggcagggtacttcgttcaagaccggcgcttggcacccttgttggaaagggattttcagcataacattttccttccacctctttgaccttccctccagcgttggccaaattgtgctgaggaagatgcatcaagggcttggctgtgccttcataggtcatctagggttttataaaggaggaggagacaatattttttcaaactttttggggagtggggtcatttctgtatataaaaaatgttaatatttaaggtgtatttatgttaccgttctgaataaacagaatggaccattgaaccagta

SEQ ID NO: 2 - protein, 782 aa, Homo sapiens
MGKRDRADRDKKKSRKRHYEDEEDDEEDAPGNDPQEAVPSAAGKQVDESGTKVDEYGAKDYRLQMPLKDDHTSRPLWVAPDGHIFLEAFSPVYKYAQDFLVAIAEPVCRPTHVHEYKLTAYSLYAAVSVGLQTSDITEYLRKLSKTGVPDGIMQFIKLCTVSYGKVKLVLKHNRYFVESCHPDVIQHLLQDPVIRECRLRNSEGEATELITETFTSKSAISKTAESSGGPSTSRVTDPQGKSDIPMDLFDFYEQMDKDEEEEEETQTVSFEVKQEMIEELQKRCIHLEYPLLAEYDFRNDSVNPDINIDLKPTAVLRPYQEKSLRKMFGNGRARSGVIVLPCGAGKSLVGVTAACTVRKRCLVLGNSAVSVEQWKAQFKMWSTIDDSQICRFTSDAKDKPIGCSVAISTYSMLGHTTKRSWEAERVMEWLKTQEWGLMILDEVHTIPAKMFRRVLTIVQAHCKLGLTATLVREDDKIVDLNFLIGPKLYEANWMELQNNGYIAKVQCAEVWCPMSPEFYREYVAIKTKKRILLYTMNPNKFRACQFLIKFHERRNDKIIVFADNVFALKEYAIRLNKPYIYGPTSQGERMQILQNFKHNPKINTIFISKVGDTSFDLPEANVLIQISSHGGSRRQEAQRLGRVLRAKKGMVAEEYNAFFYSLVSQDTQEMAYSTKRQRFLVDQGYSFKVITKLAGMEEEDLAFSTKEEQQQLLQKVLAATDLDAEEEVVAGEFGSRSSQASRRFGTMSSMSGADDTVYMEYHSSRSKAPSKHVHPLFKRFRK

SEQ ID NO: 3 - DNA, 2318 bp, Homo sapiens
atgaagctcaacgtggacgggctcctggtctacttcccgtacgactacatctaccccgagcagttctcctacatgcgggagctcaaacgcacgctggacgccaagggtcatggagtcctggagatgccctcaggcaccgggaagacagtatccctgttggccctgatcatggcataccagagagcatatccgctggaggtgaccaaactcatctactgctcaagaactgtgccagagattgagaaggtgattgaagagcttcgaaagttgctcaacttctatgagaagcaggagggcgagaagctgccgtttctgggactggctctgagctcccgcaaaaacttgtgtattcaccctgaggtgacacccctgcgctttgggaaggacgtcgatgggaaatgccacagcctcacagcctcctatgtgcgggcgcagtaccagcatgacaccagcctgccccactgccgattctatgaggaatttgatgcccatgggcgtgaggtgcccctccccgctggcatctacaacctggatgacctgaaggccctggggcggcgccagggctggtgcccatacttccttgctcgatactcaatcctgcatgccaatgtggtggtttatagctaccactacctcctggaccccaagattgcagacctggtgtccaaggaactggcccgcaaggccgtcgtggtcttcgacgaggcccacaacattgacaacgtctgcatcgactccatgagcgtcaacctcacccgccggacccttgaccggtgccagggcaacctggagaccctgcagaagacggtgctcaggatcaaagagacagacgagcagcgcctgcgggacgagtaccggcgtctggtggaggggctgcgggaggccagcgccgcccgggagacggacgcccacctggccaaccccgtgctgcccgacgaagtgctgcaggaggcagtgcctggctccatccgcacggccgagcatttcctgggcttcctgaggcggctgctggagtacgtgaagtggcggctgcgtgtgcagcatgtggtgcaggagagcccgcccgccttcctgagcggcctggcccagcgcgtgtgcatccagcgcaagcccctcagattctgtgctgaacgcctccggtccctgctgcatactctggagatcaccgaccttgctgacttctccccgctcaccctccttgctaactttgccacccttgtcagcacctacgccaaaggcttcaccatcatcatcgagccctttgacgacagaaccccgaccattgccaaccccatcctgcacttcagctgcatggacgcctcgctggccatcaaacccgtatttgagcgtttccagtctgtcatcatcacatctgggacactgtccccgctggacatctaccccaagatcctggacttccaccccgtcaccatggcaaccttcaccatgacgctggcacgggtctgcctctgccctatgatcatcggccgtggcaatgaccaggtggccatcagctccaaatttgagacccgggaggatattgctgtgatccggaactatgggaacctcctgctggagatgtccgctgtggtccctgatggcatcgtggccttcttcaccagctaccagtacatggagagcaccgtggcctcctggtatgagcaggggatccttgagaacatccagaggaacaagctgctctttattgagacccaggatggtgccgaaaccagtgtcgccctggagaagtaccaggaggcctgcgagaatggccgcggggccatcctgctgtcagtggcccggggcaaagtgtccgagggaatcgactttgtgcaccactacgggcgggccgtcatcatgtttggcgtcccctacgtctacacacagagccgcattctcaaggcgcggctggaatacctgcgggaccagttccagattcgtgagaatgactttcttaccttcgatgccatgcgccacgcggcccagtgtgtgggtcgggccatcaggggcaagacggactacggcctcatggtctttgccgacaagcggtttgcccgtggggacaagcgggggaagctgccccgctggatccaggagcacctcacagatgccaacctcaacctgaccgtggacgagggtgtccaggtggccaagtacttcctgcggcagatggcacagcccttccaccgggaggatcagctgggcctgtccctgctcagcctggagcagctagaatcagaggagacgctgaagaggatagagcagattgctcagcagctctgagtggggcgggtggggccataaacggttcctggtga

SEQ ID NO: 4 - protein, 760 aa, Homo sapiens
MKLNVDGLLVYFPYDYIYPEQFSYMRELKRTLDAKGHGVLEMPSGTGKTVSLLALIMAYQRAYPLEVTKLIYCSRTVPEIEKVIEELRKLLNFYEKQEGEKLPFLGLALSSRKNLCIHPEVTPLRFGKDVDGKCHSLTASYVRAQYQHDTSLPHCRFYEEFDAHGREVPLPAGIYNLDDLKALGRRQGWCPYFLARYSILHANVVVYSYHYLLDPKIADLVSKELARKAVVVFDEAHNIDNVCIDSMSVNLTRRTLDRCQGNLETLQKTVLRIKETDEQRLRDEYRRLVEGLREASAARETDAHLANPVLPDEVLQEAVPGSIRTAEHFLGFLRRLLEYVKWRLRVQHVVQESPPAFLSGLAQRVCIQRKPLRFCAERLRSLLHTLEITDLADFSPLTLLANFATLVSTYAKGFTIIIEPFDDRTPTIANPILHFSCMDASLAIKPVFERFQSVIITSGTLSPLDIYPKILDFHPVTMATFTMTLARVCLCPMIIGRGNDQVAISSKFETREDIAVIRNYGNLLLEMSAVVPDGIVAFFTSYQYMESTVASWYEQGILENIQRNKLLFIETQDGAETSVALEKYQEACENGRGAILLSVARGKVSEGIDFVHHYGRAVIMFGVPYVYTQSRILKARLEYLRDQFQIRENDFLTFDAMRHAAQCVGRAIRGKTDYGLMVFADKRFARGDKRGKLPRWIQEHLTDANLNLTVDEGVQVAKYFLRQMAQPFHREDQLGLSLLSLEQLESEETLKRIEQIAQQL

SEQ ID NO: 5 - DNA, 9731 bp, Artificial
  Synthesized retroviral vectors
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggagcgctcgaggcgacttacctctctagagtcggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactcccaagcttatcgaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgcatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggggcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcctgaagaaaagggagaattcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 6 - DNA, 9782 bp, Artificial
  Synthesized retroviral vectors
ccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttggatcctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgaattccaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatctgaagaaaagggaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttggcaaacctgaatagcttccagaagaagagaagtgaggataagaataacagttgtcattaaccagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt